SEQUENCE LISTING (49 sequences)

SEQ ID NO: 1 - protein, 879 aa, artificial sequence
  The PTGFRN Protein
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 2 - protein, 219 aa, artificial sequence
  Human IL-12A (p35)(signal peptide)
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 3 - protein, 328 aa, artificial sequence
  Human IL-12B (p40) (signal peptide)
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 4 - protein, 548 aa, artificial sequence
  IL-12 Fusion (signal peptide-p40-linker-p35)
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSGGGSGGGGSGGGGSGGGSGGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 5-8 - skipped (4 entries)

SEQ ID NO: 9 - protein, 385 aa, artificial sequence
  The BSG protein
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 10-13 - skipped (4 entries)

SEQ ID NO: 14 - protein, 613 aa, artificial sequence
  The IGSF8 protein
MGALRPTLLPPSLPLLLLLMLGMGCWAREVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 15-20 - skipped (6 entries)

SEQ ID NO: 21 - protein, 748 aa, artificial sequence
  The ITGB1 protein
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 22 - protein, 1032 aa, artificial sequence [sequence omitted]

SEQ ID NO: 23 - protein, 630 aa, artificial sequence
  The SLC3A2 Protein, where the first Met is processed.
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 24-32 - skipped (9 entries)

SEQ ID NO: 33 - protein, 192 aa, artificial sequence
  The PTGFRN protein Fragment
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEM

SEQ ID NO: 34-46 - skipped (13 entries)

SEQ ID NO: 47 - protein, 332 aa, artificial sequence
  The MARCKS protein
MGAQFSKTAAKGEAAAERPGEAAVASSPSKANGQENGHVKVNGDASPAAAESGAKEELQANGSAPAADKEEPAAAGSGAASPSAAEKGEPAAAAAPEAGASPVEKEAPAEGEAAEPGSPTAAEGEAASAASSTSSPKAEDGATPSPSNETPKKKKKRFSFKKSFKLSGFSFKKNKKEAGEGGEAEAPAAEGGKDEAAGGAAAAAAEAGAASGEQAAAPGEEAAAGEEGAAGGDPQEAKPQEAAVAPEKPPASDETKAAEEPSKVEEKKAEEAGASAAACEAPSAAGPGAPPEQEAAPAEEPAAAAASSACAAPSQEAQPECSPEAPPAEAAE

SEQ ID NO: 48 - protein, 195 aa, artificial sequence
  The MARCKSL1 protein
MGSQSSKAPRGDVTAEEAAGASPAKANGQENGHVKSNGDLSPKGEGESPPVNGTDEAAGATGDAIEPAPPSQGAEAKGEVPPKETPKKKKKFSFKKPFKLSGLSFKRNRKEGGGDSSASSPTEEEQEQGEIGACSDEGTAQEGKAAATPESQEPQAKGAEASAASEEEAGPQATEPSTPSGPESGPTPASAEQNE

SEQ ID NO: 49 - protein, 227 aa, artificial sequence
  The BASP1 protein
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE

(19) United States
(12) Patent Application Publication
(10) Pub. No.: US 2025/0249072 A1
(43) Pub. Date: Aug. 7, 2025

(54) METHODS OF TREATING CANCER

(71) Applicant: LONZA SALES AG, BASEL (CH)

(72) Inventors: Nuruddeen LEWIS, Andover, MA (US); Chang Ling SIA, Devens, MA (US); Katherine KIRWIN, Cambridge, MA (US); Kyriakos ECONOMIDES, Grafton, MA (US); Sriram SATHYANARAYANAN, Lexington, MA (US)

(73) Assignee: LONZA SALES AG, BASEK (CH)

(21) Appl. No.: 18/042,068

(22) PCT Filed: Aug. 17, 2021
(86) PCT No.: PCT/US2021/046358

§ 371 Date: Feb. 17, 2023

Related U.S. Application Data
(60) Provisional application No. 63/166,772, filed on Mar. 26, 2021.
(60) Provisional application No. 63/145,762, filed on Feb. 4, 2021.
(60) Provisional application No. 63/136,051, filed on Jan. 11, 2021.
(60) Provisional application No. 63/131,870, filed on Dec. 30, 2020.
(60) Provisional application No. 63/069,423, filed on Aug. 24, 2020.
(60) Provisional application No. 63/066,603, filed on Aug. 17, 2020.

(51) Int. Cl. A61K 38/20; A61K 9/00; A61K 45/06; A61K 47/64; A61P 35/00

(52) U.S. Cl. CPC A61K 38/208; A61K 9/0019; A61K 45/06; A61K 47/64; A61P 35/00

(57) ABSTRACT

The present disclosure relates to methods of treating a cancer in a subject comprising administering to the subject extracellular vesicles, e.g., exosomes, comprising a cytokine, e.g., an Interleukin-12 (IL-12) moiety.

### CROSS-REFERENCE TO EARLIER FILED APPLICATIONS

This PCT application claims the priority benefit of U.S. Provisional Application Nos. 63/166,772 filed on Mar. 26, 2021; 63/145,762 filed on Feb. 4, 2021; 63/136,051 filed on Jan. 11, 2021; 63/131,870 filed on Dec. 30, 2020; 63/069,423 filed on Aug. 24, 2020; and 63/066,603 filed on Aug. 17, 2020; each of which is incorporated by reference herein in its entirety.

The content of the electronically submitted sequence listing in ASCII text file (Name: 0132-0293US1 Sequence Listing ST25_v2.txt; Size:57,003 bytes; and Date of Creation: Oct. 27, 2023), filed with the application, is incorporated herein by reference in its entirety.

### FIELD OF DISCLOSURE

The present disclosure relates to modified extracellular vesicles, e.g., exosomes, (e.g., comprising an IL-12 moiety) that can be used to treat and/or prevent cancer. The present disclosure also relates to methods of producing such EVs, exosomes, and uses thereof.

### BACKGROUND

Immunotherapy has grown as a leading means of treating various types of cancer by inducing, enhancing, or suppressing the immune response. Immunotherapy can stimulate the patient's own immune system to attack cancer cells. Interleukin-12 (IL-12) acts as a growth factor for T cells, which can activate T cells and enhance the immune response. However, administration of free IL-12 can have far reaching effects beyond the tumor microenvironment.

EVs, exosomes, are important mediators of intercellular communication. They are also important biomarkers in the diagnosis and prognosis of many diseases. As drug delivery vehicles, EVs, e.g., exosomes offer many advantages over traditional drug delivery methods (e.g., peptide immunization, DNA vaccines) as a new treatment modality in many therapeutic areas.

### SUMMARY OF DISCLOSURE

Certain aspects of the present disclosure are directed to methods of treating cutaneous T-cell lymphoma (CTCL), triple negative breast cancer (TNBC), glioblastoma, Merkel cell carcinoma (MCC), and/or Kaposi sarcoma in a subject in need thereof comprising administering an extracellular vesicle (EV) comprising Interleukin-12 (IL-12).

Some aspects of the present disclosure are directed to methods of treating cutaneous T-cell lymphoma (CTCL) in a subject in need thereof comprising administering an extracellular vesicle (EV) comprising Interleukin-12 (IL-12). In some aspects, the CTCL is at stage IA-IIB.

Some aspects of the present disclosure are directed to methods of treating cutaneous Kaposi sarcoma in a subject in need thereof comprising administering an extracellular vesicle (EV) comprising Interleukin-12 (IL-12).

Some aspects of the present disclosure are directed to methods of treating triple negative breast cancer (TNBC) in a subject in need thereof comprising administering an extracellular vesicle (EV) comprising Interleukin-12 (IL-12).

Some aspects of the present disclosure are directed to methods of treating glioblastoma in a subject in need thereof comprising administering an extracellular vesicle (EV) comprising Interleukin-12 (IL-12).

Some aspects of the present disclosure are directed to methods of treating Merkel cell carcinoma (MCC) in a subject in need thereof comprising administering an extracellular vesicle (EV) comprising Interleukin-12 (IL-12).

In some aspects, the administration of the EV results in at least about 20%, at least about 25%, at least about 30%, at least about 35%, at least about 40%, at least about 45%, at least about 50%, at least about 55%, at least about 60%, at least about 70%, at least about 80%, at least about 90%, or about 100% objective response rate (ORR). In some aspects, the administration of the EV results in about 25-56% ORR. In some aspects, the response rate is measured using an mSWAT analysis, e.g., for CTCL.

In some aspects, the administration of the EV results in at least about 10%, at least about 20%, at least about 25%, at least about 30%, at least about 35%, at least about 40%, at least about 45%, at least about 50%, at least about 55%, at least about 60%, at least about 70%, at least about 80%, at least about 90%, or about 100% complete response (CR). In some aspects, the administration of the EV results in about 22% CR.

In some aspects, the EV further comprises a scaffold moiety. In some aspects, the IL-12 is linked to the scaffold moiety. In some aspects, the scaffold moiety is a Scaffold X. In some aspects, Scaffold X is a scaffold protein that is capable of anchoring the IL-12 on the exterior surface of the EV. In some aspects, Scaffold X is selected from the group consisting of prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), and any combination thereof.

In some aspects, the scaffold moiety is a PTGFRN protein. In some aspects, the scaffold moiety comprises an amino acid sequence as set forth in SEQ ID NO: 33. In some aspects, the scaffold moiety comprises an amino acid sequence having at least 50%, at least 60%, at least 70%, at least 80%, at least 85%, at least 90%, at least 95%, at least 96%, at least 97%, at least 98%, at least 99%, or about 100% sequence identity to SEQ ID NO: 1.

In some aspects, the IL-12 is linked to the scaffold moiety by a linker. In some aspects, the linker is a polypeptide. In some aspects, the linker is a non-polypeptide moiety.

In some aspects, the IL-12 comprises an amino acid sequence having at least 50%, at least 60%, at least 70%, at least 80%, at least 85%, at least 90%, at least 95%, at least 96%, at least 97%, at least 98%, at least 99%, or about 100% sequence identity to SEQ ID NO: 2. In some aspects, the IL-12 comprises an amino acid sequence having at least 50%, at least 60%, at least 70%, at least 80%, at least 85%, at least 90%, at least 95%, at least 96%, at least 97%, at least 98%, at least 99%, or about 100% sequence identity to SEQ ID NO: 3. In some aspects, the IL-12 is a single polypeptide.

In some aspects, the IL-12 comprises p35 and p40 linked by a linker. In some aspects, the linker is a GS linker. In some aspects, the GS linker comprises (G₄S)ₙ or (G₃S)ₙ, wherein n is any integer between 1 and 10.

In some aspects, the IL-12 comprises an amino acid sequence having at least 50%, at least 60%, at least 70%, at least 80%, at least 85%, at least 90%, at least 95%, at least 96%, at least 97%, at least 98%, at least 99%, or about 100% sequence identity to SEQ ID NO: 4.

In some aspects, the EV is administered parenterally, orally, intravenously, intramuscularly, intra-tumorally, intranasally, subcutaneously, or intraperitoneally.

In some aspects, the EV is an exosome.

Certain aspects of the present disclosure are directed to a kit comprising the EV comprising IL-12 and instructions for use according to a method disclosed herein.

In some aspects, the method further comprises administering an additional therapeutic agent. In some aspects, the additional therapeutic agent is an anti-neoplastic agent. In some aspects, the anti-neoplastic agent is an immune checkpoint inhibitor. In some aspects, the immune checkpoint inhibitor comprises an anti-PD-1 antibody, an anti-PD-L1 antibody, an anti-LAG3 antibody, an anti-TIM3 antibody, an anti-CTLA4 antibody, an anti-TIGIT antibody, or any combination thereof.

In some aspects, the EV for the present methods is administered at a therapeutically effective amount of at least about 0.3 μg, at least about 1 μg, at least about 2 μg, at least about 3 μg, at least about 4 μg, at least about 5 μg, at least about 6 μg, at least about 7 μg, at least about 8 μg, at least about 9 μg, at least about 10 μg, at least about 11 μg, or at least about 12 μg. In some aspects, the EV for the present methods is administered at a therapeutically effective amount of about 1 μg to about 12 μg, about 1 μg to about 11 μg, about 1 μg to about 10 μg, about 1 μg to about 9 μg, about 1 μg to about 8 μg, about 1 μg to about 7 μg, about 1 μg to about 6 μg, about 1 μg to about 5 μg, about 2 μg to about 12 μg, about 2 μg to about 11 μg, about 2 μg to about 10 μg, about 2 μg to about 9 μg, about 2 μg to about 8 μg, about 2 μg to about 7 μg, about 2 μg to about 6 μg, about 2 μg to about 5 μg, about 3 μg to about 12 μg, about 3 μg to about 11 μg, about 3 μg to about 10 μg, about 3 μg to about 9 μg, about 3 μg to about 8 μg, about 3 μg to about 7 μg, about 3 μg to about 6 μg, about 3 μg to about 5 μg, about 4 μg to about 12 μg, about 4 μg to about 11 μg, about 4 μg to about 10 μg, about 4 μg to about 9 μg, about 4 μg to about 8 μg, about 4 μg to about 7 μg, about 4 μg to about 6 μg, or about 4 μg to about 5 μg.

In some aspects, the EV for the present methods is administered at a therapeutically effective amount of about 0.3 μg, about 1 μg, about 2 μg, about 3 μg, about 4 μg, about 5 μg, about 6 μg, about 7 μg, about 8 μg, about 9 μg, about 10 μg, about 11 μg, or about 12 μg. In some aspects, the EV is administered at a therapeutically effective amount of about 6 μg. In some aspects, the EV is administered at a therapeutically effective amount between about 5 μg and about 7 μg, e.g., about 5 μg, about 6 μg, or about 7 μg.

In some aspects, the therapeutically effective amount of the EV in the methods is administered at least once, at least twice, or at least three times. In some aspects, the therapeutically effective amount of the EV is administered in multiple doses.

In some aspects, the therapeutically effective amount of the EV exhibits less systemic toxicity in the subject compared to the administration of the same dose of recombinant IL-12.

In some aspects, the EV is administered at a therapeutically effective amount between about 5 μg and about 7 μg, e.g., about 6 μg, once about every week, once about every other week, once about every three weeks, or once about every four weeks. In some aspects, the EV is administered at a therapeutically effective amount between about 5 μg and about 7 μg, e.g., about 6 μg, once about every other week. In some aspects, the EV is administered at a therapeutically effective amount between about 5 μg and about 7 μg, e.g., about 6 μg, once about every 10 to 18 days, once about every 12 to about 16 days, once about every 14 to about 21 days, once about every 10 to 14 days, or once about every 14 to about 18 days. In some aspects, the EV is administered at a therapeutically effective amount between about 5 μg and about 7 μg, e.g., about 6 μg, once about every 14 days.

### BRIEF DESCRIPTION OF FIGURES

FIG. 1 is a drawing of an engineered exosome having surface displayed IL-12 (“exoIL-12”).

FIGS. 2A-2D are scatter plots showing tumor retention (FIG. 2A), IFN-gamma AUC (FIG. 2B), tumor growth (FIG. 2C), and tumor growth following rechallenge in untreated mice and mice treated with free recombinant IL-12 or exoIL-12, as indicated. FIG. 2E is a box-plot showing the percent of antigen-specific CD8⁺ T cells in control and exoIL-12-treated mice.

FIGS. 3A-3C are graphical representations the pharmacokinetics, as measured by exoIL-12 measured in the skin and plasma (FIG. 3A), and tissue pharmacodynamics, as measured by the fold change over vehicle of IP-10 in the skin (FIG. 3B) and plasma (FIG. 3C) over time, following administration of 0.3 μg, 1.0 μg, or 3.0 μg exoIL-12.

FIGS. 4A-4B are schematic representations of a clinical study assessing the safety and efficacy of exoIL-12 treatment in healthy volunteers (FIG. 4A) and cancer patients (FIG. 4B).

FIGS. 5A-5B are schematic illustrations of plasma (FIG. 5A) and skin (FIG. 5B) sample biopsies.

FIG. 6 is a graphical representation of plasma IL-12 (μg/mL) concentration in human subjects administered 0.3 μg, 1.0 μg, 3.0 μg, 6.0 μg, or 12.0 μg exoIL-12 up to 700 hours after administration. All data from each dosing amount are pooled.

FIGS. 7A-7D are graphical representations of IP-10 (FIGS. 7A-7C) and IL-12 (FIG. 7D) levels in the skin (FIGS. 7A and 7C) and plasma (FIGS. 7B and 7D) of subjects administered 0.3 μg, 1.0 μg, 3.0 μg, 6.0 μg, or 12.0 μg exoIL-12.

FIG. 8 is a graphical representation of plasma IFNγ (μg/mL) concentration in human subjects administered 0.3 μg (mcg), 1.0 μg, 3.0 μg, 6.0 μg, or 12.0 μg exoIL-12 up to 700 hours after administration.

### DETAILED DESCRIPTION OF DISCLOSURE

The present disclosure is directed to methods of treating a cancer in a subject in need thereof, comprising administering an EV, e.g., exosome, comprising a cytokine, e.g., an IL-12 moiety, to the subject. In some aspects, the cytokine, e.g., IL-12 moiety, is attached (or linked) to one or more scaffold moieties on the surface of the EV, e.g., exosome, or on the luminal surface of the EV, e.g., exosome. In some aspects, the cancer is selected from the group consisting of cutaneous T-cell lymphoma (CTCL), melanoma, triple negative breast cancer (TNBC), glioblastoma, Merkel cell carcinoma (MCC), and/or Kaposi sarcoma.

In some aspects, the subject is not administered an EV, e.g., exosome, engineered to comprise a STING agonist.

Non-limiting examples of the various aspects are shown in the present disclosure.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, and U represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The headings provided herein are not limitations of the various aspects of the disclosure, which can be had by reference to the specification as a whole. Accordingly, the terms defined immediately below are more fully defined by reference to the specification in its entirety.

The term “about” or “approximately” is used herein to mean approximately, roughly, around, or in the region of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some aspects, the term used herein means within 5% of the referenced amount, e.g., about 50% is understood to encompass a range of values from 47.5% to 52.5%.

As used herein, the term “extracellular vesicle” or “EV” refers to a cell-derived vesicle comprising a membrane that encloses an internal space. Extracellular vesicles comprise all membrane-bound vesicles (e.g., exosomes, nanovesicles) that have a smaller diameter than the cell from which they are derived. Generally, extracellular vesicles range in diameter from 20 nm to 1000 nm, and can comprise various macromolecular payload either within the internal space (i.e., lumen), displayed on the external surface of the extracellular vesicle, and/or spanning the membrane. In some aspects, the payload can comprise nucleic acids, proteins, carbohydrates, lipids, small molecules, and/or combinations thereof. In certain aspects, an extracellular vesicle comprises a scaffold moiety. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are produced by cells that express one or more transgene products.

As used herein the term “exosome” refers to a cell-derived small (between 20-300 nm in diameter, more preferably 40-200 nm in diameter) vesicle comprising a membrane that encloses an internal space (i.e., lumen), and which is generated from said cell by direct plasma membrane budding or by fusion of the late endosome with the plasma membrane. The exosome is a species of extracellular vesicle. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. In some aspects, an exosome comprises a scaffold moiety. The exosome can be derived from a producer cell, and isolated from the producer cell based on its size, density, biochemical parameters, or a combination thereof. In some aspects, the exosomes of the present disclosure are produced by cells that express one or more transgene products.

As used herein, the term “nanovesicle” refers to a cell-derived small (between 20-250 nm in diameter, more preferably 30-150 nm in diameter) vesicle comprising a membrane that encloses an internal space, and which is generated from said cell by direct or indirect manipulation such that said nanovesicle would not be produced by said producer cell without said manipulation. Appropriate manipulations of said producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. In some aspects, a nanovesicle comprises a scaffold moiety. The nanovesicle, once it is derived from a producer cell according to said manipulation, may be isolated from the producer cell based on its size, density, biochemical parameters, or a combination thereof.

As used herein the term “surface-engineered EVs, e.g., exosomes” (e.g., Scaffold X-engineered EVs, e.g., exosomes) refers to an EV, e.g., exosome, with the membrane or the surface of the EV, e.g., exosome, modified in its composition so that the surface of the engineered EV, e.g., exosome, is different from that of the EV, e.g., exosome, prior to the modification or of the naturally occurring EV, e.g., exosome. The engineering can be on the surface of the EV, e.g., exosome, or in the membrane of the EV, e.g., exosome, so that the surface of the EV, e.g., exosome, is changed. For example, the membrane is modified in its composition of a protein, a lipid, a small molecule, a carbohydrate, etc. The composition can be changed by a chemical, a physical, or a biological method or by being produced from a cell previously or concurrently modified by a chemical, a physical, or a biological method. Specifically, the composition can be changed by a genetic engineering or by being produced from a cell previously modified by genetic engineering. In some aspects, a surface-engineered EV, e.g., exosome, comprises an exogenous protein (i.e., a protein that the EV, e.g., exosome, does not naturally express) or a fragment or variant thereof that can be exposed to the surface of the EV, e.g., exosome, or can be an anchoring point (attachment) for a moiety exposed on the surface of the EV, e.g., exosome. In other aspects, a surface-engineered EV, e.g., exosome, comprises a higher expression (e.g., higher number) of a natural exosome protein (e.g., Scaffold X) or a fragment or variant thereof that can be exposed to the surface of the EV, e.g., exosome, or can be an anchoring point (attachment) for a moiety exposed on the surface of the EV, e.g., exosome.

As used herein the term “lumen-engineered exosome” (e.g., Scaffold Y-engineered exosome) refers to an EV, e.g., exosome, with the membrane or the lumen of the EV, e.g., exosome, modified in its composition so that the lumen of the engineered EV, e.g., exosome, is different from that of the EV, e.g., exosome, prior to the modification or of the naturally occurring EV, e.g., exosome. The engineering can be directly in the lumen or in the membrane of the EV, e.g., exosome so that the lumen of the EV, e.g., exosome is changed. For example, the membrane is modified in its composition of a protein, a lipid, a small molecule, a carbohydrate, etc. so that the lumen of the EV, e.g., exosome is modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some aspects, a lumen-engineered exosome comprises an exogenous protein (i.e., a protein that the EV, e.g., exosome does not naturally express) or a fragment or variant thereof that can be exposed in the lumen of the EV, e.g., exosome or can be an anchoring point (attachment) for a moiety exposed on the inner layer of the EV, e.g., exosome. In other aspects, a lumen-engineered EV, e.g., exosome, comprises a higher expression of a natural exosome protein (e.g., Scaffold X or Scaffold Y) or a fragment or variant thereof that can be exposed to the lumen of the exosome or can be an anchoring point (attachment) for a moiety exposed in the lumen of the exosome.

The term “modified,” when used in the context of EVs, e.g., exosomes described herein, refers to an alteration or engineering of an EV, e.g., exosome and/or its producer cell, such that the modified EV, e.g., exosome is different from a naturally-occurring EV, e.g., exosome. In some aspects, a modified EV, e.g., exosome described herein comprises a membrane that differs in composition of a protein, a lipid, a small molecular, a carbohydrate, etc. compared to the membrane of a naturally-occurring EV, e.g., exosome (e.g., membrane comprises higher density or number of natural exosome proteins and/or membrane comprises proteins that are not naturally found in exosomes (e.g., An IL-12 moiety). In certain aspects, such modifications to the membrane changes the exterior surface of the EV, e.g., exosome (e.g., surface-engineered EVs, e.g., exosomes described herein). In certain aspects, such modifications to the membrane changes the lumen of the EV, e.g., exosome (e.g., lumen-engineered EVs, e.g., exosomes described herein).

As used herein, the term “scaffold moiety” refers to a molecule that can be used to anchor an TL-12 moiety or any other compound of interest to the EV, e.g., exosome, either on the luminal surface or on the exterior surface of the EV, e.g., exosome. In certain aspects, a scaffold moiety comprises a synthetic molecule. In some aspects, a scaffold moiety comprises a non-polypeptide moiety. In other aspects, a scaffold moiety comprises a lipid, carbohydrate, or protein that naturally exists in the EV, e.g., exosome. In some aspects, a scaffold moiety comprises a lipid, carbohydrate, or protein that does not naturally exist in the EV, e.g., exosome. In certain aspects, a scaffold moiety is Scaffold X. In some aspects, a scaffold moiety is Scaffold Y. In further aspects, a scaffold moiety comprises both Scaffold X and Scaffold Y. In certain aspects, a scaffold moiety comprises Lamp-1, Lamp-2, CD13, CD86, Flotillin, Syntaxin-3, CD2, CD36, CD40, CD40L, CD41a, CD44, CD45, ICAM-1, Integrin alpha4, L1CAM, LFA-1, Mac-1 alpha and beta, Vti-1A and B, CD3 epsilon and zeta, CD9, CD18, CD37, CD53, CD63, CD81, CD82, CXCR4, FcR, GluR2/3, HLA-DM (MHC II), immunoglobulins, MHC-I or MHC-II components, TCR beta, tetraspanins, or combinations thereof.

As used herein, the term “Scaffold X” refers to exosome proteins that have recently been identified on the surface of exosomes. See, e.g., U.S. Pat. No. 10,195,290, which is incorporated herein by reference in its entirety. Non-limiting examples of Scaffold X proteins include: prostaglandin F2 receptor negative regulator (“the PTGFRN protein”); basigin (“the BSG protein”); immunoglobulin superfamily member 2 (“the IGSF2 protein”); immunoglobulin superfamily member 3 (“the IGSF3 protein”); immunoglobulin superfamily member 8 (“the IGSF8 protein”); integrin beta-1 (“the ITGB1 protein); integrin alpha-4 (“the ITGA4 protein”); 4F2 cell-surface antigen heavy chain (“the SLC3A2 protein”); and a class of ATP transporter proteins (“the ATP1A1 protein,” “the ATP1A2 protein,” “the ATP1A3 protein,” “the ATP1A4 protein,” “the ATP1B3 protein,” “the ATP2B1 protein,” “the ATP2B2 protein,” “the ATP2B3 protein,” “the ATP2B protein”). In some aspects, a Scaffold X protein can be a whole protein or a fragment thereof (e.g., functional fragment, e.g., the smallest fragment that is capable of anchoring another moiety on the exterior surface or on the luminal surface of the EV, e.g., exosome). In some aspects, a Scaffold X can anchor a moiety (e.g., an IL-12 moiety) to the external surface or the luminal surface of the exosome.

As used herein, the term “Scaffold Y” refers to exosome proteins that were newly identified within the luminal surface of exosomes. See, e.g., International Publication. No. WO/2019/099942, which is incorporated herein by reference in its entirety. Non-limiting examples of Scaffold Y proteins include: myristoylated alanine rich Protein Kinase C substrate (“the MARCKS protein”); myristoylated alanine rich Protein Kinase C substrate like 1 (“the MARCKSL1 protein”); and brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety on the luminal surface of the EVs, e.g., exosomes). In some aspects, a Scaffold Y can anchor an TL-2 moiety to the lumen of the EVs, e.g., exosomes.

As used herein, the term “fragment” of a protein (e.g., therapeutic protein, Scaffold X, or Scaffold Y) refers to an amino acid sequence of a protein that is shorter than the naturally-occurring sequence, N- and/or C-terminally deleted or any part of the protein deleted in comparison to the naturally occurring protein. As used herein, the term “functional fragment” refers to a protein fragment that retains protein function. Accordingly, in some aspects, a functional fragment of a Scaffold X protein retains the ability to anchor a moiety on the luminal surface or on the exterior surface of the EV, e.g., exosome. Similarly, in certain aspects, a functional fragment of a Scaffold Y protein retains the ability to anchor a moiety on the luminal surface of the EV, e.g., exosome. Whether a fragment is a functional fragment can be assessed by any art known methods to determine the protein content of EVs, e.g., exosomes including Western Blots, FACS analysis and fusions of the fragments with autofluorescent proteins like, e.g., GFP. In certain aspects, a functional fragment of a Scaffold X protein retains at least about 50%, at least about 60%, at least about 70%, at least about 80%, at least about 90% or at least about 100% of the ability, e.g., an ability to anchor a moiety, of the naturally occurring Scaffold X protein. In some aspects, a functional fragment of a Scaffold Y protein retains at least about 50%, at least about 60%, at least about 70%, at least about 80%, at least about 90% or at least about 100% of the ability, e.g., an ability to anchor another molecule, of the naturally occurring Scaffold Y protein.

As used herein, the term “variant” of a molecule (e.g., functional molecule, antigen, Scaffold X and/or Scaffold Y) refers to a molecule that shares certain structural and functional identities with another molecule upon comparison by a method known in the art. For example, a variant of a protein can include a substitution, insertion, deletion, frameshift or rearrangement in another protein.

In some aspects, a variant of a Scaffold X comprises a variant having at least about 70% identity to the full-length, mature PTGFRN, BSG, IGSF2, IGSF3, IGSF8, ITGB1, ITGA4, SLC3A2, or ATP transporter proteins or a fragment (e.g., functional fragment) of the PTGFRN, BSG, IGSF2, IGSF3, IGSF8, ITGB1, ITGA4, SLC3A2, or ATP transporter proteins. In some aspects, variants or variants of fragments of PTGFRN share at least about 70%, at least about 80%, at least about 85%, at least about 90%, at least about 95%, at least about 96%, at least about 97%, at least about 98%, or at least about 99% sequence identity with PTGFRN according to SEQ ID NO: 1 or with a functional fragment thereof.

In some aspects, a variant of a Scaffold Y comprises a variant having at least 70% identity to MARCKS, MARCKSL1, BASP1, or a fragment of MARCKS, MARCKSL1, or BASP1. In some aspects variants or variants of fragments of MARCKS share at least about 70%, at least about 80%, at least about 85%, at least about 90%, at least about 95%, at least about 96%, at least about 97%, at least about 98%, or at least about 99% sequence identity with MARCKS according to SEQ ID NO: 47 or with a functional fragment thereof. In some aspects variants or variants of fragments of MARCKSL1 share at least about 70%, at least about 80%, at least about 85%, at least about 90%, at least about 95%, at least about 96%, at least about 97%, at least about 98%, or at least about 99% sequence identity with MARCKSL1 according to SEQ ID NO: 48 or with a functional fragment thereof. In some aspects variants or variants of fragments of BASP1 share at least about 70%, at least about 80%, at least about 85%, at least about 90%, at least about 95%, at least about 96%, at least about 97%, at least about 98%, or at least about 99% sequence identity with BASP1 according to SEQ ID NO: 49 or with a functional fragment thereof. In some aspects, the variant or variant of a fragment of Scaffold Y protein retains the ability to be specifically targeted to the lumen of EVs, e.g., exosomes. In some aspects, the Scaffold Y includes one or more mutations, e.g., conservative amino acid substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences may be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity may be curated either automatically or manually.

The polynucleotide variants can contain alterations in the coding regions, non-coding regions, or both. In one aspect, the polynucleotide variants contain alterations which produce silent substitutions, additions, or deletions, but do not alter the properties or activities of the encoded polypeptide. In another aspect, nucleotide variants are produced by silent substitutions due to the degeneracy of the genetic code. In other aspects, variants in which 5-10, 1-5, or 1-2 amino acids are substituted, deleted, or added in any combination. Polynucleotide variants can be produced for a variety of reasons, e.g., to optimize codon expression for a particular host (change codons in the human mRNA to others, e.g., a bacterial host such as E. coli).

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-la. They used random mutagenesis to generate over 3,500 individual IL-la mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, polypeptide variants include, e.g., modified polypeptides. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination. In some aspects, Scaffold X and/or Scaffold Y is modified at any convenient location.

As used herein the term “linked to” or “conjugated to” are used interchangeably and refer to a covalent or non-covalent bond formed between a first moiety and a second moiety, e.g., Scaffold X and an IL-12 moiety, respectively, e.g., a scaffold moiety expressed in or on the extracellular vesicle and an IL-12 moiety, e.g., Scaffold X (e.g., a PTGFRN protein), respectively, in the luminal surface of or on the external surface of the extracellular vesicle.

The term “encapsulated”, or grammatically different forms of the term (e.g., encapsulation, or encapsulating) refers to a status or process of having a first moiety (e.g., an IL-12 moiety) inside a second moiety (e.g., an EV, e.g., exosome) without chemically or physically linking the two moieties. In some aspects, the term “encapsulated” can be used interchangeably with “in the lumen of”. Non-limiting examples of encapsulating a first moiety (e.g., an IL-12 moiety) into a second moiety (e.g., EVs, e.g., exosomes) are disclosed elsewhere herein.

As used herein, the term “producer cell” refers to a cell used for generating an EV, e.g., exosome. A producer cell can be a cell cultured in vitro, or a cell in vivo. A producer cell includes, but not limited to, a cell known to be effective in generating EVs, e.g., exosomes, e.g., HEK293 cells, Chinese hamster ovary (CHO) cells, mesenchymal stem cells (MSCs), BJ human foreskin fibroblast cells, s9f cells, fHDF fibroblast cells, AGE.N® neuronal precursor cells, CAP® amniocyte cells, adipose mesenchymal stem cells, and RPTEC/TERT1 cells. In certain aspects, a producer cell is an antigen-presenting cell. In some aspects, the producer cell is a bacterial cell. In some aspects, a producer cell is a dendritic cell, a B cell, a mast cell, a macrophage, a neutrophil, a Kupffer-Browicz cell, or a cell derived from any of these cells, or any combination thereof. In some aspects, the producer cell is not a bacterial cell. In other aspects, the producer cell is not an antigen-presenting cell.

As used herein the term “associated with” refers to a covalent or non-covalent bond formed between a first moiety, e.g., an IL-12 moiety, and a second moiety, e.g., an extracellular vesicle, respectively; or encapsulation of a first moiety, e.g., an IL-12 moiety, into a second moiety, e.g., extracellular vesicle. For example, in some aspects, a scaffold moiety, e.g., Scaffold X (e.g., a PTGFRN protein), is expressed in or on the extracellular vesicle and an IL-12 moiety, is loaded on the external surface of the extracellular vesicle. In one aspect, the term “associated with” means a covalent, non-peptide bond or a non-covalent bond. For example, the amino acid cysteine comprises a thiol group that can form a disulfide bond or bridge with a thiol group on a second cysteine residue. Examples of covalent bonds include, but are not limited to, a peptide bond, a metal bond, a hydrogen bond, a disulfide bond, a sigma bond, a pi bond, a delta bond, a glycosidic bond, an agnostic bond, a bent bond, a dipolar bond, a Pi backbond, a double bond, a triple bond, a quadruple bond, a quintuple bond, a sextuple bond, conjugation, hyperconjugation, aromaticity, hapticity, or antibonding. Non-limiting examples of non-covalent bond include an ionic bond (e.g., cation-pi bond or salt bond), a metal bond, a hydrogen bond (e.g., dihydrogen bond, dihydrogen complex, low-barrier hydrogen bond, or symmetric hydrogen bond), van der Walls force, London dispersion force, a mechanical bond, a halogen bond, aurophilicity, intercalation, stacking, entropic force, or chemical polarity. In other aspects, the term “associated with” means that a first moiety, e.g., extracellular vesicle, encapsulates a second moiety, e.g., an IL-12 moiety. In some aspects, the first moiety and the second moiety can be linked to each other. In other aspects, the first moiety and the second moiety are not physically and/or chemically linked to each other.

As used herein the term “linked to” or “conjugated to” are used interchangeably and refer to a covalent or non-covalent bond formed between a first moiety and a second moiety, e.g., an IL-12 moiety and an extracellular vesicle, respectively. In some aspects, a scaffold moiety is expressed in or on the extracellular vesicle, e.g., Scaffold X (e.g., a PTGFRN protein), and an IL-12 moiety is linked to or conjugated to the portion of the Scaffold X protein (e.g., the PTGFRN protein) that is exposed on the surface of the extracellular vesicle (e.g., “surface-display of IL-12”). In some aspects, a scaffold moiety is expressed in or on the extracellular vesicle, e.g., Scaffold X (e.g., a PTGFRN protein), and an IL-12 moiety is linked to or conjugated to the portion of the Scaffold X protein (e.g., the PTGFRN protein) that is exposed to the lumen of the extracellular vesicle.

The term “loaded”, or grammatically different forms of the term (e.g., load or loaded), as used herein, refers to a status or process of having a first moiety (e.g., an IL-12 moiety) associated with a second moiety (e.g., an EV, e.g., and exosome). In some aspects, the first moiety is chemically or physically linked to the second moiety. In some aspects, the first moiety is not chemically or physically linked to the second moiety. In some aspects, the first moiety is present within the second moiety, e.g., within the lumen of an EV (e.g., an exosome), e.g., “encapsulated”. In some aspects, the first moiety is associated with the exterior surface of the second moiety, e.g., linked or conjugated to the surface of an EV (e.g., an exosome), e.g., “surface-display” of the second moiety.

The term “encapsulated”, or grammatically different forms of the term (e.g., encapsulation, or encapsulating), refers to a status or process of having a first moiety (e.g., an IL-12 moiety) inside a second moiety (e.g., an EV, e.g., exosome) without chemically or physically linking the two moieties. In some aspects, the term “encapsulated” can be used interchangeably with “in the lumen of”. Non-limiting examples of encapsulating a first moiety (e.g., an IL-12 moiety) into a second moiety (e.g., EVs, e.g., exosomes) are disclosed elsewhere herein.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) of the EVs from a sample containing producer cells. In some aspects, an isolated EV composition has no detectable undesired activity or, alternatively, the level or amount of the undesired activity is at or below an acceptable level or amount. In other aspects, an isolated EV composition has an amount and/or concentration of desired EVs at or above an acceptable amount and/or concentration. In other aspects, the isolated EV composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some aspects, isolated EV preparations are substantially free of residual biological products. In some aspects, the isolated EV preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

The term “free IL-12 moiety” as used herein means an IL-12 moiety that is not associated with an extracellular vesicle, but otherwise identical to the IL-12 moiety associated with the extracellular vesicle. Especially when compared to an extracellular vesicle associated with an IL-12 moiety, the free IL-12 moiety is the same IL-12 moiety associated with the extracellular vesicle. In some aspects, when a free IL-12 moiety is compared to an extracellular vesicle comprising the IL-12 moiety in its efficacy, toxicity, and/or any other characteristics, the amount of the free IL-12 moiety compared to the IL-12 moiety associated with the extracellular vesicle is the same as the amount of the IL-12 moiety associated with the EV.

The term “exoIL-12” as used herein refers to an exosome loaded with an IL-12 moiety, e.g., an IL-12 protein or a fragment thereof. In some aspects, the IL-12 moiety is associated with the exterior surface of the exosome (e.g., surface display of the IL-12 moiety). In some aspects, the IL-12 moiety is linked to or conjugated to the exterior surface of the exosome. In some aspects, the IL-12 moiety is linked to or conjugated to a surface exposed scaffold protein, e.g., a Scaffold X protein, e.g., a PTGFRN protein. In some aspects, the IL-12 moiety is linked to or conjugated to the lipid bilayer of the exosome. In some aspects, the exosome comprises an IL-12 moiety in the lumen of the exosome. In some aspects, the IL-12 moiety is associated with the luminal surface of the exosome, e.g., with a Scaffold protein, e.g., Scaffold X, e.g., PTGFRN. In some aspects, the IL-12 moiety is encapsulated within the lumen of the exosome and is not associated with a scaffold protein.

As used herein, the term “ligand” refers to a molecule that binds to a receptor and modulates the receptor to produce a biological response. Modulation can be activation, deactivation, blocking, or damping of the biological response mediated by the receptor. Receptors can be modulated by either an endogenous or an exogenous ligand. Non-limiting examples of endogenous ligands include antibodies and peptides. Non-limiting examples of exogenous agonist include drugs, small molecules, and cyclic dinucleotides. The ligand can be a full, partial, or inverse ligand.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. As used herein, the term “antigen” refers to any agent that when introduced into a subject elicits an immune response (cellular or humoral) to itself. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function.

As used herein the term “therapeutically effective amount” is the amount of reagent or pharmaceutical compound that is sufficient to a produce a desired therapeutic effect, pharmacologic and/or physiologic effect on a subject in need thereof. A therapeutically effective amount can be a “prophylactically effective amount” as prophylaxis can be considered therapy.

As used herein, the term “pharmaceutical composition” refers to one or more of the compounds described herein, such as, e.g., an EV mixed or intermingled with, or suspended in one or more other chemical components, such as pharmaceutically-acceptable carriers and excipients. One purpose of a pharmaceutical composition is to facilitate administration of preparations of EVs to a subject. The term “excipient” or “carrier” refers to an inert substance added to a pharmaceutical composition to further facilitate administration of a compound. The term “pharmaceutically-acceptable carrier” or “pharmaceutically-acceptable excipient” and grammatical variations thereof, encompasses any of the agents approved by a regulatory agency of the US Federal government or listed in the US Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “payload” refers to a therapeutic agent that acts on a target (e.g., a target cell) that is contacted with the EV. Payloads that can be introduced into an EV and/or a producer cell include therapeutic agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety or a toxin or that disrupt translation), polypeptides (e.g., enzymes), lipids, carbohydrates, and small molecules (e.g., small molecule drugs and toxins).

The terms “administration,” “administering” and variants thereof refer to introducing a composition, such as an EV, or agent into a subject and includes concurrent and sequential introduction of a composition or agent. The introduction of a composition or agent into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

The terms “individual,” “subject,” “host,” and “patient,” are used interchangeably herein and refer to any mammalian subject for whom diagnosis, treatment, or therapy is desired, particularly humans. The compositions and methods described herein are applicable to both human therapy and veterinary applications. In some aspects, the subject is a mammal, and in other aspects the subject is a human. As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

As used herein, the term “substantially free” means that the sample comprising EVs, e.g., exosomes, comprise less than 10% of macromolecules by mass/volume (m/v) percentage concentration. Some fractions may contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules.

As used herein, the term “macromolecule” means nucleic acids, contaminant proteins, lipids, carbohydrates, metabolites, or a combination thereof.

As used herein, the term “conventional exosome protein” means a protein previously known to be enriched in exosomes, including but is not limited to CD9, CD63, CD81, PDGFR, GPI anchor proteins, lactadherin (MFGE8), LAMP2, and LAMP2B, a fragment thereof, or a peptide that binds thereto.

An “immune response,” as used herein, refers to a biological response within a vertebrate against foreign agents or abnormal, e.g., infected cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell. Accordingly, an immune response can comprise a humoral immune response (e.g., mediated by B-cells), cellular immune response (e.g., mediated by T cells), or both humoral and cellular immune responses. In some aspects, an immune response is an “inhibitory” immune response. An inhibitory immune response is an immune response that blocks or diminishes the effects of a stimulus (e.g., antigen). In certain aspects, the inhibitory immune response comprises the production of inhibitory antibodies against the stimulus. In some aspects, the inhibitory response comprises the production of antibodies against the cytokine, e.g., antibodies against IL-12. In some aspects, an immune response is a “stimulatory” immune response. A stimulatory immune response is an immune response that results in the generation of effectors cells (e.g., cytotoxic T lymphocytes) that can destroy and clear a target antigen (e.g., a tumor antigen).

“Treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also includes prophylaxis or prevention of a disease or condition or its symptoms thereof. In one aspect, the term “treating” or “treatment” means inducing an immune response in a subject against an antigen. In some aspects, the disease or condition is a cancer. In some aspects, the cancer is selected from the group consisting of cutaneous T-cell lymphoma (CTCL), triple negative breast cancer (TNBC), glioblastoma, Merkel cell carcinoma (MCC), Kaposi sarcoma, or any combination thereof. In some aspects, the cancer comprises melanoma.

“Prevent” or “preventing,” as used herein, refers to decreasing or reducing the occurrence or severity of a particular outcome. In some aspects, preventing an outcome is achieved through prophylactic treatment. In some aspects, an EV, e.g., an exosome, comprising a cytokine, e.g., an IL-12 moiety, described herein is administered to a subject prophylactically. In some aspects, the subject is at risk of developing a cancer selected from the group consisting of cutaneous T-cell lymphoma (CTCL), triple negative breast cancer (TNBC), melanoma, glioblastoma, Merkel cell carcinoma (MCC), Kaposi sarcoma, or any combination thereof.

As used herein, the term “modulate,” “modulating”, “modify,” and/or “modulator” generally refers to the ability to alter, by increase or decrease, e.g., directly or indirectly promoting/stimulating/up-regulating or interfering with/inhibiting/down-regulating a specific concentration, level, expression, function or behavior, such as, e.g., to act as an antagonist or agonist. In some instances a modulator can increase and/or decrease a certain concentration, level, activity or function relative to a control, or relative to the average level of activity that would generally be expected or relative to a control level of activity.

As used herein, “a mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

The terms “individual,” “subject,” “host,” and “patient,” are used interchangeably herein and refer to any mammalian subject for whom diagnosis, treatment, or therapy is desired, particularly humans. The methods described herein are applicable to both human therapy and veterinary applications. In some aspects, the subject is a mammal, and in other aspects the subject is a human.

A “primary tumor,” as used herein, refers to an original, or first, tumor in a subject, where the tumor initiated growth. A primary tumor is used in contrast to a “secondary tumor,” which refers to a tumor that arises after initiation of growth of the primary tumor at a location other than the location of the primary tumor, e.g., due to metastasis of cells in the primary tumor.

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### II. Methods of the Disclosure

Certain aspects of the present disclosure are directed to methods of treating a cancer in a subject in need thereof, comprising administering an EV, e.g., an exosome, comprising a cytokine, e.g., IL-12, to the subject. In some aspects, the cancer is selected from the group consisting of cutaneous T-cell lymphoma (CTCL), melanoma, triple negative breast cancer (TNBC), glioblastoma, Merkel cell carcinoma (MCC), Kaposi sarcoma, and any combination thereof. In some aspects, the cancer comprises a bladder cancer. In some aspects, the cancer comprises a basal cell carcinoma (BCC). In some aspects, the EV comprises IL-12, e.g., human IL-12. In some aspects, the subject is not administered an EV, e.g., exosome, engineered to comprise a STING agonist.

In some aspects, the exosomes described herein comprise an IL-12 moiety. In certain aspects, the IL-12 moiety comprises human IL-12 or a fragment thereof. Human IL-12 is an interleukin that is naturally produced by dendritic cells, macrophages, neutrophils, and B-lymphoblastoid cells. IL-12 is known to stimulate the growth and function of T cells, including stimulating the production of IFN-gamma and TNF-alpha and reducing IL-4-mediated suppression of IFN-gamma. IL-12 is also involved in the differentiation of naïve T cells into Th1 cells. IL-12 also enhances the lytic activity of NK/lymphokine-activated killer cells. IL-12 is a heterodimeric cytokine, composed of an IL-12A (p35) subunit and an IL-12B (p40) subunit. The active heterodimer is also known as p70. The amino acid sequences for IL-12A (p35; Uniprot P29459; SEQ TD NO: 2) and IL-12B (p40; Uniprot P29460; SEQ ID NO: 3) are shown in Table 1.

TABLE 1
IL-12 Amino Acid Sequences.

| | |
|---|---|
| Human IL- | MCPARSLLLVATLVLLDHLSLARNLPVATPDPGMFPCLHHSQNLLRAVSNMLQKA |
| 12A | R |
| (p35) (signal | QTLEFYPCTSEEIDHEDITKDKTSTVEACLPLELTKNESCLNSRETSFITNGSCL |
| peptide) | A |
| SEQ ID NO: | SRKTSFMMALCLSSIYEDLKMYQVEFKTMNAKLLMDPKRQIFLDQNMLAVIDELM |
| 2 | Q |
| | ALNFNSETVPQKSSLEEPDFYKTKIKLCILLHAFRIRAVTIDRVMSYLNAS |
| Human IL- | MCHQQLVISWFSLVFLASPLVAIWELKKDVYVVELDWYPDAPGEMVVLTCDTPEE |
| 12B (p40) | D |
| (signal | GITWTLDQSSEVLGSGKTLTIQVKEFGDAGQYTCHKGGEVLSHSLLLLHKKEDGI |
| peptide) | W |
| SEQ ID NO: | STDILKDQKEPKNKTFLRCEAKNYSGRFTCWWLTTISTDLTFSVKSSRGSSDPQG |
| 3 | V |
| | TCGAATLSAERVRGDNKEYEYSVECQEDSACPAAEESLPIEVMVDAVHKLKYENY |
| | T |
| | SSFFIRDIIKPDPPKNLQLKPLKNSRQVEVSWEYPDTWSTPHSYFSLTFCVQVQG |
| | K |
| | SKREKKDRVFTDKTSATVICRKNASISVRAQDRYYSSSWSEWASVPCS |
| IL-12 | MCHQQLVISWFSLVFLASPLVAIWELKKDVYVVELDWYPDAPGEMVVLTCDTPEE |
| Fusion | D |
| (signal | GITWTLDQSSEVLGSGKTLTIQVKEFGDAGQYTCHKGGEVLSHSLLLLHKKEDGI |
| peptide- | W |
| p40-linker- | STDILKDQKEPKNKTFLRCEAKNYSGRFTCWWLTTISTDLTFSVKSSRGSSDPQG |
| p35) | V |
| SEQ ID NO: | TCGAATLSAERVRGDNKEYEYSVECQEDSACPAAEESLPIEVMVDAVHKLKYENY |
| 4 | T |
| | SSFFIRDIIKPDPPKNLQLKPLKNSRQVEVSWEYPDTWSTPHSYFSLTFCVQVQG |
| | K |
| | SKREKKDRVFTDKTSATVICRKNASISVRAQDRYYSSSWSEWASVPCSGGSGGGS |
| | G |
| | GGGSGGGGSGGGSGGRNLPVATPDPGMFPCLHHSQNLLRAVSNMLQKARQTLEFY |
| | P |
| | CTSEEIDHEDITKDKTSTVEACLPLELTKNESCLNSRETSFITNGSCLASRKTSF |
| | M |
| | MALCLSSIYEDLKMYQVEFKTMNAKLLMDPKRQIFLDQNMLAVIDELMQALNFNS |
| | E |
| | TVPQKSSLEEPDFYKTKIKLCILLHAFRIRAVTIDRVMSYLNAS |

In some aspects, the IL-12 moiety comprises a p35 polypeptide or a fragment thereof. In some aspects, the IL-12 comprises an amino acid sequence having at least about 50%, at least about 60%, at least about 70%, at least about 80%, at least about 85%, at least about 90%, at least about 95%, at least about 96%, at least about 97%, at least about 98%, at least about 99%, or about 100% sequence identity to SEQ ID NO: 2. In some aspects, the IL-12 comprises an amino acid sequence having at least about 90% sequence identity to SEQ TD NO: 2. In some aspects, the IL-12 comprises an amino acid sequence having at least about 95% sequence identity to SEQ ID NO: 2. In some aspects, the IL-12 comprises an amino acid sequence having at least about 96% sequence identity to SEQ ID NO: 2. In some aspects, the IL-12 comprises an amino acid sequence having at least about 97% sequence identity to SEQ ID NO: 2. In some aspects, the IL-12 comprises an amino acid sequence having at least about 98% sequence identity to SEQ ID NO: 2. In some aspects, the IL-12 comprises an amino acid sequence having at least about 99% sequence identity to SEQ ID NO: 2. In some aspects, the IL-12 comprises the amino acid sequence set forth in SEQ ID NO: 2. In some aspects, the IL-12 moiety lacks a signal peptide (see Table 1).

In some aspects, the IL-12 moiety comprises a p40 polypeptide or a fragment thereof. In some aspects, the IL-12 comprises an amino acid sequence having at least about 50%, at least about 60%, at least about 70%, at least about 80%, at least about 85%, at least about 90%, at least about 95%, at least about 96%, at least about 97%, at least about 98%, at least about 99%, or about 100% sequence identity to SEQ ID NO: 3. In some aspects, the IL-12 comprises an amino acid sequence having at least about 90% sequence identity to SEQ ID NO: 3. In some aspects, the IL-12 comprises an amino acid sequence having at least about 95% sequence identity to SEQ ID NO: 3. In some aspects, the IL-12 comprises an amino acid sequence having at least about 96% sequence identity to SEQ ID NO: 3. In some aspects, the IL-12 comprises an amino acid sequence having at least about 97% sequence identity to SEQ ID NO: 3. In some aspects, the IL-12 comprises an amino acid sequence having at least about 98% sequence identity to SEQ ID NO: 3. In some aspects, the IL-12 comprises an amino acid sequence having at least about 99% sequence identity to SEQ ID NO: 3. In some aspects, the IL-12 comprises the amino acid sequence set forth in SEQ ID NO: 3. In some aspects, the IL-12 moiety lacks a signal peptide (see Table 1).

In some aspects, the IL-12 moiety comprises a p35 polypeptide or a fragment thereof and a p40 polypeptide or a fragment thereof. In some aspects, IL-12 moiety comprises a single polypeptide, wherein the p35 polypeptide or a fragment thereof is linked to the p40 polypeptide or a fragment thereof. In some aspects, the p35 polypeptide or a fragment thereof is linked to the p40 polypeptide or a fragment thereof by a linker. In some aspects, the linker is a peptide linker. In some aspects, the linker comprises one or more amino acids. In some aspects, the linker comprises a Gly-Ser (GS) linker. In some aspects, the GS linker comprises (G₄S)ₙ, wherein n is an integer between 1 and 10. In some aspects, the GS linker comprises (G₃S)ₙ, wherein n is an integer between 1 and 10.

In certain aspects, the IL-12 moiety comprises an amino acid sequence having at least about 50%, at least about 60%, at least about 70%, at least about 80%, at least about 85%, at least about 90%, at least about 95%, at least about 96%, at least about 97%, at least about 98%, at least about 99%, or about 100% sequence identity to SEQ ID NO: 4. In some aspects, the IL-12 comprises an amino acid sequence having at least about 90% sequence identity to SEQ ID NO: 4. In some aspects, the IL-12 comprises an amino acid sequence having at least about 95% sequence identity to SEQ ID NO: 4. In some aspects, the IL-12 comprises an amino acid sequence having at least about 96% sequence identity to SEQ ID NO: 4. In some aspects, the IL-12 comprises an amino acid sequence having at least about 97% sequence identity to SEQ ID NO: 4. In some aspects, the IL-12 comprises an amino acid sequence having at least about 98% sequence identity to SEQ ID NO: 4. In some aspects, the IL-12 comprises an amino acid sequence having at least about 99% sequence identity to SEQ ID NO: 4. In some aspects, the IL-12 comprises the amino acid sequence set forth in SEQ ID NO: 4. In some aspects, the IL-12 consists of the amino acid sequence set forth in SEQ ID NO: 4. In some aspects, the IL-12 consists essentially of the amino acid sequence set forth in SEQ ID NO: 4. In some aspects, the IL-12 moiety lacks a signal peptide (see Table 1).

In some aspects, the administration of the EV results in an objective response rate (ORR) of about 20% to about 100%, about 30% to about 100%, about 40% to about 100%, about 50% to about 100%, about 60% to about 100%, about 70% to about 100%, or about 80% to about 100%. In some aspects, the administration of the EV results in an ORR of about 20% to about 90%, about 20% to about 80%, about 20% to about 75%, about 20% to about 70%, about 20% to about 65%, about 20% to about 60%, about 20% to about 55%, about 20% to about 50%. In some aspects, the administration of the EV results in an ORR of about 25% to about 56%. In some aspects, the administration of the EV results in at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% ORR. In some aspects, the response rate is measured using an mSWAT analysis, e.g., for CTCL.

In some aspects, the administration of the EV results in a complete response in the subject. In some aspects, the administration of the EV results in at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of subjects experiencing a complete response (CR). In some aspects, the administration of the EV results in at least about 20% of subjects experiencing a CR. In some aspects, the administration of the EV results in at least about 22% of subjects experiencing a CR.

Without being bound by any particular mechanism, in some aspects, administration of the EVs disclosed herein activates immune cells that target and kill tumor cells. In some aspects, the EVs disclosed herein, e.g., EVs loaded with an IL-12 moiety, activate T cells. In some aspects, the EVs disclosed herein, e.g., EVs loaded with an IL-12 moiety, activate CD8⁺ T cells. In some aspects, the EVs disclosed herein, e.g., EVs loaded with an IL-12 moiety, activate NK cells.

The EVs can be administered to a subject by any route known in the art. In some aspects, the EVs are administered parenterally, orally, intravenously (IV), intramuscularly (IM), intra-tumorally (IT), intranasally, subcutaneously, or intraperitoneally (IP). In certain aspects, the EVs are administered directly into the tumor microenvironment, e.g., by IT delivery. In some aspects, IT delivery of the EVs disclosed herein reduces systemic exposure, enhances selective immune cell activation, increases tissue retention (providing a prolonged response), or any combination thereof.

In some aspects, the EV for the present methods is administered at a therapeutically effective amount of at least about 0.3 μg, at least about 1 μg, at least about 2 μg, at least about 3 μg, at least about 4 μg, at least about 5 μg, at least about 6 μg, at least about 7 μg, at least about 8 μg, at least about 9 μg, at least about 10 μg, at least about 11 μg, or at least about 12 μg. In some aspects, the EV for the present methods is administered at a therapeutically effective amount of at least about 0.3 μg. In some aspects, the EV for the present methods is administered at a therapeutically effective amount of at least about 1 μg. In some aspects, the EV for the present methods is administered at a therapeutically effective amount of at least about 2 μg. In some aspects, the EV for the present methods is administered at a therapeutically effective amount of at least about 3 μg. In some aspects, the EV for the present methods is administered at a therapeutically effective amount of at least about 4 μg. In some aspects, the EV for the present methods is administered at a therapeutically effective amount of at least about 5 μg. In some aspects, the EV for the present methods is administered at a therapeutically effective amount of at least about 6 μg. In some aspects, the EV for the present methods is administered at a therapeutically effective amount of at least about 7 μg. In some aspects, the EV for the present methods is administered at a therapeutically effective amount of at least about 8 μg. In some aspects, the EV for the present methods is administered at a therapeutically effective amount of at least about 9 μg. In some aspects, the EV for the present methods is administered at a therapeutically effective amount of at least about 10 μg. In some aspects, the EV for the present methods is administered at a therapeutically effective amount of at least about 11 μg. In some aspects, the EV for the present methods is administered at a therapeutically effective amount of at least about 12 μg.

In some aspects, the EV for the present methods is administered at a therapeutically effective amount of about 0.3 μg to about 12 μg, about 1 μg to about 12 μg, about 1 μg to about 11 μg, about 1 μg to about 10 μg, about 1 μg to about 9 μg, about 1 μg to about 8 μg, about 1 μg to about 7 μg, about 1 μg to about 6 μg, about 1 μg to about 5 μg, about 2 μg to about 12 μg, about 2 μg to about 11 μg, about 2 μg to about 10 μg, about 2 μg to about 9 μg, about 2 μg to about 8 μg, about 2 μg to about 7 μg, about 2 μg to about 6 μg, about 2 μg to about 5 μg, about 3 μg to about 12 μg, about 3 μg to about 11 μg, about 3 μg to about 10 μg, about 3 μg to about 9 μg, about 3 μg to about 8 μg, about 3 μg to about 7 μg, about 3 μg to about 6 μg, about 3 μg to about 5 μg, about 4 μg to about 12 μg, about 4 μg to about 11 μg, about 4 μg to about 10 μg, about 4 μg to about 9 μg, about 4 μg to about 8 μg, about 4 μg to about 7 μg, about 4 μg to about 6 μg, or about 4 μg to about 5 μg.

In some aspects, the EV for the present methods is administered at a therapeutically effective amount of about 1 μg to about 2 μg, about 2 μg to about 3 μg, about 3 μg to about 4 μg, about 4 μg to about 5 μg, about 5 μg to about 6 μg, about 6 μg to about 7 μg, about 7 μg to about 8 μg, about 8 μg to about 9 μg, about 9 μg to about 10 μg, about 10 μg to about 11 μg, or about 11 μg to about 12 μg. In some aspects, the EV for the present methods is administered at a therapeutically effective amount of about 1 μg to about 2 μg. In some aspects, the EV for the present methods is administered at a therapeutically effective amount of about 2 μg to about 3 μg. In some aspects, the EV for the present methods is administered at a therapeutically effective amount of about 3 μg to about 4 μg. In some aspects, the EV for the present methods is administered at a therapeutically effective amount of about 4 μg to about 5 μg. In some aspects, the EV for the present methods is administered at a therapeutically effective amount of about 5 μg to about 6 μg. In some aspects, the EV for the present methods is administered at a therapeutically effective amount of about 6 μg to about 7 μg. In some aspects, the EV for the present methods is administered at a therapeutically effective amount of about 7 μg to about 8 μg. In some aspects, the EV for the present methods is administered at a therapeutically effective amount of about 8 μg to about 9 μg. In some aspects, the EV for the present methods is administered at a therapeutically effective amount of about 9 μg to about 10 μg. In some aspects, the EV for the present methods is administered at a therapeutically effective amount of about 10 μg to about 11 μg. In some aspects, the EV for the present methods is administered at a therapeutically effective amount of about 11 μg to about 12 μg.

In some aspects, the EV for the present methods is administered at a therapeutically effective amount of about 0.3 μg, about 1 μg, about 2 μg, about 3 μg, about 4 μg, about 5 μg, about 6 μg, about 7 μg, about 8 μg, about 9 μg, about 10 μg, about 11 μg, or about 12 μg. In some aspects, the EV for the present methods is administered at a therapeutically effective amount of about 0.3 μg. In some aspects, the EV for the present methods is administered at a therapeutically effective amount of about 1 μg. In some aspects, the EV for the present methods is administered at a therapeutically effective amount of about 2 μg. In some aspects, the EV for the present methods is administered at a therapeutically effective amount of about 3 μg. In some aspects, the EV for the present methods is administered at a therapeutically effective amount of about 4 μg. In some aspects, the EV for the present methods is administered at a therapeutically effective amount of about 5 μg. In some aspects, the EV for the present methods is administered at a therapeutically effective amount of about 6 μg. In some aspects, the EV for the present methods is administered at a therapeutically effective amount of about 7 μg. In some aspects, the EV for the present methods is administered at a therapeutically effective amount of about 8 μg. In some aspects, the EV for the present methods is administered at a therapeutically effective amount of about 9 μg. In some aspects, the EV for the present methods is administered at a therapeutically effective amount of about 10 μg. In some aspects, the EV for the present methods is administered at a therapeutically effective amount of about 11 μg. In some aspects, the EV for the present methods is administered at a therapeutically effective amount of about 12 μg.

In some aspects, the therapeutically effective amount of the EV in the methods is administered at least once, at least twice, or at least three times. In some aspects, the therapeutically effective amount of the EV is administered in multiple doses.

In some aspects, the therapeutically effective amount of the EV exhibits less systemic toxicity in the subject compared to the administration of the same dose of recombinant IL-12.

In some aspects, the EV is administered once about every week, once about every other week, once about every three weeks, or once about every four weeks. In some aspects, the EV is administered once about every other week. In some aspects, the EV is administered once about every 7 to 21 days, once about every 7 to 18 days, once about every 7 to 14 days, once about every 10 to 21 days, once about every 10 to 18 days, once about every 10 to 14 days, once about every 10 to 16 days, once about every 12 to about 20 days, once about every 12 to about 18 days, once about every 12 to about 16 days, once about every 12 to about 14 days, once about every 14 to about 21 days, once about every 14 to about 16 days, once about every 14 to about 18 days, or once about every 14 to about 21 days. In some aspects, the EV is administered once about every 7 days. In some aspects, the EV is administered once about every 10 days. In some aspects, the EV is administered once about every 12 days. In some aspects, the EV is administered once about every 14 days. In some aspects, the EV is administered once about every 16 days. In some aspects, the EV is administered once about every 18 days. In some aspects, the EV is administered once about every 20 days. In some aspects, the EV is administered once about every 21 days.

In some aspects, the EV is administered at a therapeutically effective amount between about 5 μg and about 7 μg, e.g., about 6 μg, once about every week, once about every other week, once about every three weeks, or once about every four weeks. In some aspects, the EV is administered at a therapeutically effective amount between about 5 μg and about 7 μg, e.g., about 6 μg, once about every other week. In some aspects, the EV is administered at a therapeutically effective amount between about 5 μg and about 7 μg, e.g., about 6 μg, once about every 10 to 18 days, once about every 12 to about 16 days, once about every 14 to about 21 days, once about every 10 to 14 days, or once about every 14 to about 18 days. In some aspects, the EV is administered at a therapeutically effective amount between about 5 μg and about 7 μg, e.g., about 6 μg, once about every 14 days.

In some aspects, the EV is administered at a therapeutically effective amount of about 3.0 μg once about every week. In some aspects, the EV is administered at a therapeutically effective amount of about 3.0 μg once about every 7 days. In some aspects, the EV is administered at a therapeutically effective amount of about 3.0 μg once about every 8 days. In some aspects, the EV is administered at a therapeutically effective amount of about 3.0 μg once about every 9 days. In some aspects, the EV is administered at a therapeutically effective amount of about 3.0 μg once about every 10 days. In some aspects, the EV is administered at a therapeutically effective amount of about 3.0 μg once about every 11 days. In some aspects, the EV is administered at a therapeutically effective amount of about 3.0 μg once about every 12 days. In some aspects, the EV is administered at a therapeutically effective amount of about 3.0 μg once about every 13 days. In some aspects, the EV is administered at a therapeutically effective amount of about 3.0 μg once about every 14 days. In some aspects, the EV is administered at a therapeutically effective amount of about 3.0 μg once about every 2 weeks. In some aspects, the EV is administered at a therapeutically effective amount of about 3.0 μg once about every 15 days. In some aspects, the EV is administered at a therapeutically effective amount of about 3.0 μg once about every 15 days. In some aspects, the EV is administered at a therapeutically effective amount of about 3.0 μg once about every 17 days. In some aspects, the EV is administered at a therapeutically effective amount of about 3.0 μg once about every 18 days. In some aspects, the EV is administered at a therapeutically effective amount of about 3.0 μg once about every 19 days. In some aspects, the EV is administered at a therapeutically effective amount of about 3.0 μg once about every 20 days. In some aspects, the EV is administered at a therapeutically effective amount of about 3.0 μg once about every 21 days. In some aspects, the EV is administered at a therapeutically effective amount of about 3.0 μg once about every 3 weeks. In some aspects, the EV is administered at a therapeutically effective amount of about 3.0 μg once about every week. In some aspects, the EV is administered at a therapeutically effective amount of about 3.0 μg once about every 7 days. In some aspects, the EV is administered at a therapeutically effective amount of about 3.0 μg once about every 8 days. In some aspects, the EV is administered at a therapeutically effective amount of about 3.0 μg once about every 9 days. In some aspects, the EV is administered at a therapeutically effective amount of about 3.0 μg once about every 10 days. In some aspects, the EV is administered at a therapeutically effective amount of about 3.0 μg once about every 11 days. In some aspects, the EV is administered at a therapeutically effective amount of about 3.0 μg once about every 12 days. In some aspects, the EV is administered at a therapeutically effective amount of about 3.0 μg once about every 13 days. In some aspects, the EV is administered at a therapeutically effective amount of about 3.0 μg once about every 14 days. In some aspects, the EV is administered at a therapeutically effective amount of about 3.0 μg once about every 2 weeks. In some aspects, the EV is administered at a therapeutically effective amount of about 3.0 μg once about every 15 days. In some aspects, the EV is administered at a therapeutically effective amount of about 3.0 μg once about every 15 days. In some aspects, the EV is administered at a therapeutically effective amount of about 3.0 μg once about every 17 days. In some aspects, the EV is administered at a therapeutically effective amount of about 3.0 μg once about every 18 days. In some aspects, the EV is administered at a therapeutically effective amount of about 3.0 μg once about every 19 days. In some aspects, the EV is administered at a therapeutically effective amount of about 3.0 μg once about every 20 days. In some aspects, the EV is administered at a therapeutically effective amount of about 3.0 μg once about every 21 days. In some aspects, the EV is administered at a therapeutically effective amount of about 3.0 μg once about every 3 weeks.

In some aspects, the EV is administered at a therapeutically effective amount of about 6.0 μg once about every week. In some aspects, the EV is administered at a therapeutically effective amount of about 6.0 μg once about every 7 days. In some aspects, the EV is administered at a therapeutically effective amount of about 6.0 μg once about every 8 days. In some aspects, the EV is administered at a therapeutically effective amount of about 6.0 μg once about every 9 days. In some aspects, the EV is administered at a therapeutically effective amount of about 6.0 μg once about every 10 days. In some aspects, the EV is administered at a therapeutically effective amount of about 6.0 μg once about every 11 days. In some aspects, the EV is administered at a therapeutically effective amount of about 6.0 μg once about every 12 days. In some aspects, the EV is administered at a therapeutically effective amount of about 6.0 μg once about every 13 days. In some aspects, the EV is administered at a therapeutically effective amount of about 6.0 μg once about every 14 days. In some aspects, the EV is administered at a therapeutically effective amount of about 6.0 μg once about every 2 weeks. In some aspects, the EV is administered at a therapeutically effective amount of about 6.0 μg once about every 15 days. In some aspects, the EV is administered at a therapeutically effective amount of about 6.0 μg once about every 15 days. In some aspects, the EV is administered at a therapeutically effective amount of about 6.0 μg once about every 17 days. In some aspects, the EV is administered at a therapeutically effective amount of about 6.0 μg once about every 18 days. In some aspects, the EV is administered at a therapeutically effective amount of about 6.0 μg once about every 19 days. In some aspects, the EV is administered at a therapeutically effective amount of about 6.0 μg once about every 20 days. In some aspects, the EV is administered at a therapeutically effective amount of about 6.0 μg once about every 21 days. In some aspects, the EV is administered at a therapeutically effective amount of about 6.0 μg once about every 3 weeks. In some aspects, the EV is administered at a therapeutically effective amount of about 6.0 μg once about every week. In some aspects, the EV is administered at a therapeutically effective amount of about 6.0 μg once about every 7 days. In some aspects, the EV is administered at a therapeutically effective amount of about 6.0 μg once about every 8 days. In some aspects, the EV is administered at a therapeutically effective amount of about 6.0 μg once about every 9 days. In some aspects, the EV is administered at a therapeutically effective amount of about 6.0 μg once about every 10 days. In some aspects, the EV is administered at a therapeutically effective amount of about 6.0 μg once about every 11 days. In some aspects, the EV is administered at a therapeutically effective amount of about 6.0 μg once about every 12 days. In some aspects, the EV is administered at a therapeutically effective amount of about 6.0 μg once about every 13 days. In some aspects, the EV is administered at a therapeutically effective amount of about 6.0 μg once about every 14 days. In some aspects, the EV is administered at a therapeutically effective amount of about 6.0 μg once about every 2 weeks. In some aspects, the EV is administered at a therapeutically effective amount of about 6.0 μg once about every 15 days. In some aspects, the EV is administered at a therapeutically effective amount of about 6.0 μg once about every 15 days. In some aspects, the EV is administered at a therapeutically effective amount of about 6.0 μg once about every 17 days. In some aspects, the EV is administered at a therapeutically effective amount of about 6.0 μg once about every 18 days. In some aspects, the EV is administered at a therapeutically effective amount of about 6.0 μg once about every 19 days. In some aspects, the EV is administered at a therapeutically effective amount of about 6.0 μg once about every 20 days. In some aspects, the EV is administered at a therapeutically effective amount of about 6.0 μg once about every 21 days. In some aspects, the EV is administered at a therapeutically effective amount of about 6.0 μg once about every 3 weeks.

In some aspects, the EV is administered at a therapeutically effective amount of about 12.0 μg once about every week. In some aspects, the EV is administered at a therapeutically effective amount of about 12.0 μg once about every 7 days. In some aspects, the EV is administered at a therapeutically effective amount of about 12.0 μg once about every 8 days. In some aspects, the EV is administered at a therapeutically effective amount of about 12.0 μg once about every 9 days. In some aspects, the EV is administered at a therapeutically effective amount of about 12.0 μg once about every 10 days. In some aspects, the EV is administered at a therapeutically effective amount of about 12.0 μg once about every 11 days. In some aspects, the EV is administered at a therapeutically effective amount of about 12.0 μg once about every 12 days. In some aspects, the EV is administered at a therapeutically effective amount of about 12.0 μg once about every 13 days. In some aspects, the EV is administered at a therapeutically effective amount of about 12.0 μg once about every 14 days. In some aspects, the EV is administered at a therapeutically effective amount of about 12.0 μg once about every 2 weeks. In some aspects, the EV is administered at a therapeutically effective amount of about 12.0 μg once about every 15 days. In some aspects, the EV is administered at a therapeutically effective amount of about 12.0 μg once about every 15 days. In some aspects, the EV is administered at a therapeutically effective amount of about 12.0 μg once about every 17 days. In some aspects, the EV is administered at a therapeutically effective amount of about 12.0 μg once about every 18 days. In some aspects, the EV is administered at a therapeutically effective amount of about 12.0 μg once about every 19 days. In some aspects, the EV is administered at a therapeutically effective amount of about 12.0 μg once about every 20 days. In some aspects, the EV is administered at a therapeutically effective amount of about 12.0 μg once about every 21 days. In some aspects, the EV is administered at a therapeutically effective amount of about 12.0 μg once about every 3 weeks. In some aspects, the EV is administered at a therapeutically effective amount of about 12.0 μg once about every 22 days. In some aspects, the EV is administered at a therapeutically effective amount of about 12.0 μg once about every 23 days. In some aspects, the EV is administered at a therapeutically effective amount of about 12.0 μg once about every 24 days. In some aspects, the EV is administered at a therapeutically effective amount of about 12.0 μg once about every 25 days. In some aspects, the EV is administered at a therapeutically effective amount of about 12.0 μg once about every 26 days. In some aspects, the EV is administered at a therapeutically effective amount of about 12.0 μg once about every 27 days. In some aspects, the EV is administered at a therapeutically effective amount of about 12.0 μg once about every 28 days. In some aspects, the EV is administered at a therapeutically effective amount of about 12.0 μg once about every 4 weeks. In some aspects, the EV is administered at a therapeutically effective amount of about 12.0 μg once about every month.

In some aspects, the EV is administered at a therapeutically effective amount of about 3.0 μg once about every 2 weeks for at least about 1 dose, at least about 2 doses, at least about 3 doses, at least about 4 doses, at least about 5 doses, at least about 6 doses, at least about 7 doses, at least about 8 doses, at least about 9 doses, at least about 10 doses, at least about 11 doses, at least about 12 doses, at least about 13 doses, at least about 14 doses, at least about 15 doses, at least about 16 doses, at least about 17 doses, at least about 18 doses, at least about 19 doses, at least about 20 doses, at least about 25 doses, at least about 30 doses, at least about 35 doses, at least about 40 doses, at least about 45 doses, or at least about 50 doses. In some aspects, the EV is administered at a therapeutically effective amount of about 3.0 μg once about every 2 weeks for at least about 2 weeks, at least about 4 weeks, at least about weeks, at least about 6 weeks, at least about 8 weeks, at least about 10 weeks, at least about 12 weeks, at least about 14 weeks, at least about 16 weeks, at least about 18 weeks, at least about 20 weeks, at least about 22 weeks, at least about 24 weeks, at least about 26 weeks, at least about 28 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks, or for at least about 52 weeks. In some aspects, the EV is administered at a therapeutically effective amount of about 3.0 μg once about every 2 weeks for at least about 6 weeks. In some aspects, the EV is administered at a therapeutically effective amount of about 3.0 μg once about every 2 weeks for at least about 12 weeks. In some aspects, the EV is administered at a therapeutically effective amount of about 3.0 μg once about every 2 weeks for as long as therapeutically effective.

In some aspects, the EV is administered at a therapeutically effective amount of about 6.0 μg once about every 2 weeks for at least about 1 dose, at least about 2 doses, at least about 3 doses, at least about 4 doses, at least about 5 doses, at least about 6 doses, at least about 7 doses, at least about 8 doses, at least about 9 doses, at least about 10 doses, at least about 11 doses, at least about 12 doses, at least about 13 doses, at least about 14 doses, at least about 15 doses, at least about 16 doses, at least about 17 doses, at least about 18 doses, at least about 19 doses, at least about 20 doses, at least about 25 doses, at least about 30 doses, at least about 35 doses, at least about 40 doses, at least about 45 doses, or at least about 50 doses. In some aspects, the EV is administered at a therapeutically effective amount of about 6.0 μg once about every 2 weeks for at least about 2 weeks, at least about 4 weeks, at least about weeks, at least about 6 weeks, at least about 8 weeks, at least about 10 weeks, at least about 12 weeks, at least about 14 weeks, at least about 16 weeks, at least about 18 weeks, at least about 20 weeks, at least about 22 weeks, at least about 24 weeks, at least about 26 weeks, at least about 28 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks, or for at least about 52 weeks. In some aspects, the EV is administered at a therapeutically effective amount of about 6.0 μg once about every 2 weeks for at least about 6 weeks. In some aspects, the EV is administered at a therapeutically effective amount of about 6.0 μg once about every 2 weeks for at least about 12 weeks. In some aspects, the EV is administered at a therapeutically effective amount of about 6.0 μg once about every 2 weeks for as long as therapeutically effective.

In some aspects, the EV is administered at a therapeutically effective amount of about 12.0 μg once about every 2 weeks for at least about 1 dose, at least about 2 doses, at least about 3 doses, at least about 4 doses, at least about 5 doses, at least about 6 doses, at least about 7 doses, at least about 8 doses, at least about 9 doses, at least about 10 doses, at least about 11 doses, at least about 12 doses, at least about 13 doses, at least about 14 doses, at least about 15 doses, at least about 16 doses, at least about 17 doses, at least about 18 doses, at least about 19 doses, at least about 20 doses, at least about 25 doses, at least about 30 doses, at least about 35 doses, at least about 40 doses, at least about 45 doses, or at least about 50 doses. In some aspects, the EV is administered at a therapeutically effective amount of about 12.0 μg once about every 2 weeks for at least about 2 weeks, at least about 4 weeks, at least about weeks, at least about 6 weeks, at least about 8 weeks, at least about 10 weeks, at least about 12 weeks, at least about 14 weeks, at least about 16 weeks, at least about 18 weeks, at least about 20 weeks, at least about 22 weeks, at least about 24 weeks, at least about 26 weeks, at least about 28 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks, or for at least about 52 weeks. In some aspects, the EV is administered at a therapeutically effective amount of about 12.0 μg once about every 2 weeks for at least about 6 weeks. In some aspects, the EV is administered at a therapeutically effective amount of about 12.0 μg once about every 2 weeks for at least about 12 weeks. In some aspects, the EV is administered at a therapeutically effective amount of about 12.0 μg once about every 2 weeks for as long as therapeutically effective.

### II.A. Cutaneous T-Cell Lymphoma

Certain aspects of the present disclosure are directed to methods of treating cutaneous T-cell lymphoma (CTCL) in a subject in need thereof comprising administering an extracellular vesicle (EV) comprising an IL-12 moiety. As used herein, CTCL refers to a type of non-Hodgkin T-cell lymphoma that affects the skin. There are several subtypes of CTCL, including but not limited to mycosis fungoides (MF), Sezary syndrome (SS), and a spectrum of T-cell neoplasms referred to as CD30-positive lymphoproliferative disorders. As such, in certain aspects, the methods disclosed herein treat an MF in a subject in need thereof. In some aspects, the methods disclosed herein treat an SS in a subject in need thereof. In some aspects, the methods disclosed herein treat a CD30-positive lymphoproliferative disorder.

In some aspects, the CTCL comprises an aggressive phenotype. In some aspects, the CTCL comprises an indolent, i.e., slow growing, phenotype.

There are four main stages of CTCL, e.g., MF, SS, and/or a CD30-positive lymphoproliferative disorder. Stage 1 CTCL is characterized by a phenotype that only affects the skin, presenting as patches or plaques. During stage 1A, less than one tenth of the skin is affected; and during stage 1B, one tenth or more of the skin is affected. Stage 2 CTCL is characterized by patches or plaques on the skin and enlarged lymph nodes, which lack abnormal lymphoma cells (stage 2A); or presentation of one or more raised lumps or tumors on the skin, with or without enlarged lymph nodes (stage 2B). Stage 3 CTCL is characterized by patches or plaques affecting four-fifths or more of the skin, with general redness, swelling, itching, and sometimes pain (erythroderma) of the skin; enlarged lymph nodes, which lack abnormal lymphoma cells; and either few or no lymphoma cells in the bloodstream (erythrodermic mycosis fungoides; Stage 3A) or moderate numbers of lymphoma cells in the bloodstream (Sezary syndrome; Stage 3B). Stage 4 CTCL affects the skin and has also spread to the bloodstream, lymph nodes, or other organs. Stage 4A refers to presentation of numerous abnormal lymphoma cells in the bloodstream (Sezary syndrome) or lymphoma cells in the lymph nodes; and Stage 4B refers to the spread of the lymphoma to other organs.

In some aspects, the methods disclosed herein treat a stage A CTCL. In some aspects, the methods disclosed herein treat a stage 1B CTCL. In some aspects, the methods disclosed herein treat a stage 2A CTCL. In some aspects, the methods disclosed herein treat a stage 2B CTCL. In some aspects, the methods disclosed herein treat a stage 3A CTCL. In some aspects, the methods disclosed herein treat a stage 3B CTCL. In some aspects, the methods disclosed herein treat a stage 4A CTCL. In some aspects, the methods disclosed herein treat a stage 4B CTCL.

### II.B. Merkel Cell Carcinoma

Certain aspects of the present disclosure are directed to methods of treating Merkel cell carcinoma (MCC) in a subject in need thereof comprising administering an extracellular vesicle (EV) comprising an IL-12 moiety. As used herein, MCC, or neuroendocrine carcinoma of the skin, refers to a rare type of skin cancer characterized by a flesh-colored or bluish-red nodule, often presenting on the face, head or neck. MCC is generally an aggressive form of skin cancer that has a high risk for recurrence and metastasis.

Standard of care therapy for MCC includes radiotherapy, surgery, systemic therapies or chemotherapy, or a combination thereof. In addition, antibodies that inhibit the interaction of programmed death 1 (PD-1) and its ligand (PD-L1) have been approved for the treatment of MCC, including avelumab.

Stage 0 MCC refers to presentation of abnormal Merkel cells in the top layer of the skin. Stage 1 MCC refers to presentation of a tumor that is 2 cm or smaller in diameter. Stage 2 MCC refers to either a tumor that is larger than 2 cm (stage 2A) or to a tumor that has spread to nearby connective tissue, muscle, cartilage, or bone (stage 2B). Stage 3A MCC refers to (i) a tumor of any size that has spread to nearby connective tissue, muscle, cartilage, or bone, and wherein cancer is found in the lymph node; or (ii) spread to lymph node can be observed by physical exam and confirmed by biopsy. Stage 3B MCC refers to presentation of a tumor of any size, which has (i) spread to nearby connective tissue, muscle, cartilage, or bone and wherein cancer is found in the lymph node; or (ii) spread to a lymph vessel between the primary tumor and proximal and/or distal lymph nodes. Stage IV MCC refers to metastasis of the cancer to a distal location on the skin or to other organs, e.g., liver, lung, bone, or brain.

In some aspects, the methods disclosed herein treat a stage 0 MCC. In some aspects, the methods disclosed herein treat a stage 1 MCC. In some aspects, the methods disclosed herein treat a stage 2A MCC. In some aspects, the methods disclosed herein treat a stage 2B MCC. In some aspects, the methods disclosed herein treat a stage 3A MCC. In some aspects, the methods disclosed herein treat a stage 3B MCC. In some aspects, the methods disclosed herein treat a stage 4 MCC. In some aspects, the methods disclosed herein treat recurrent MCC.

### II.C. Kaposi Sarcoma

Certain aspects of the present disclosure are directed to methods of treating Kaposi sarcoma in a subject in need thereof comprising administering an extracellular vesicle (EV) comprising an IL-12 moiety. As used herein, Kaposi sarcoma refers to a cancer that develops from the cells that line lymph or blood vessels, which usually appears as tumors (purple, red, or brown blotches) on the skin, on mucosal surfaces such as inside the mouth, or less frequently in other parts of the body including in the lymph nodes.

There are at least four types of Kaposi sarcomas: epidemic Kaposi sarcoma (which develops in people infected with HIV), classic Kaposi sarcoma, endemic Kaposi sarcoma, and iatrogenic (transplant-related) Kaposi sarcoma. Often, Kaposi sarcoma is associated with a weakened immune system, e.g., in malnourished or elderly patients or patients suffering from a chronic viral infection (e.g., HIV or HSV).

Kaposi sarcoma is typically treated by immune reconstitution, in particular for HIV patients. Chemotherapy can also be administered, including but not limited to doxorubicin, daunorubicine, paclitaxel, and combinations thereof.

### II.D. Triple-Negative Breast Cancer

Certain aspects of the present disclosure are directed to methods of treating triple-negative breast cancer (TNBC) in a subject in need thereof comprising administering an extracellular vesicle (EV) comprising an IL-12 moiety. As used herein, TNBC refers to a breast cancer characterized by tumors that test negative for expression of estrogen receptor (ER-negative), progesterone receptor (PR-negative), and HER2 (HER2-negative). TNBC is considered an aggressive cancer, which grows quickly, which is more likely to have spread at the time of diagnosis, and which a higher incidence of relapse after therapy than other forms of breast cancer. As a result, TNBC has a lower survival rate than other breast cancers, with the 5-year survival rate for localized TNBC being about 91%, regional TNBC being about 65%, and distant TNBC being about 11%.

As TNBC lacks expression of ER, PR, and HER2, there are limited treatments available for TNBC. For non-metastatic TNBC, chemotherapy followed by surgery is the primary treatment option. Metastatic TNBC is typically treated using chemotherapy (e.g., PARP inhibitors, platinum-based chemotherapies, and/or immunotherapies).

Stage 0 TNBC refers to non-invasive breast cancers, such as DCIS (ductal carcinoma in situ). In stage 0, there is no evidence of cancer cells or non-cancerous abnormal cells breaking out of the part of the breast in which they started, or getting through to or invading neighboring normal tissue. Stage I TNBC refers to invasive breast cancer (cancer cells are observed breaking through to or invading normal surrounding breast tissue). Stage 1A refers to invasive breast cancer in which the tumor measures up to 2 centimeters (cm) and the cancer has not spread outside the breast (e.g., the lymph nodes are not affected). Stage 1B refers to invasive breast cancer in which (i) small groups of cancer cells (0.2 mm to 2 mm in diameter) are found in the lymph nodes but there is no tumor presenting in the breast, or (ii) there is a tumor presenting in the breast that is no larger than 2 cm, and there are small groups of cancer cells (0.2 mm to 2 mm in diameter) in the lymph nodes. Microscopic invasion is possible in stage 1 breast cancer. In microscopic invasion, the cancer cells have just started to invade the tissue outside the lining of the duct or lobule, but the invading cancer cells do not measure more than 1 mm. For HER2-negative breast cancers (e.g., TNBC), a tumor that is larger than 2 cm but not larger than 5 cm and that has not spread to the lymph nodes is considered as stage 1. Stage 2A refers to a breast cancer wherein (i) no tumor can be found in the breast, but cancer (larger than 2 mm) is found in one to three axillary lymph nodes or in the lymph nodes near the breast bone; or (ii) the tumor measures 2 cm or smaller and has spread to the axillary lymph nodes. Stage 2B refers to invasive breast cancers in which (i) the tumor is 2 cm to 5 cm in size, with presentation of small groups of breast cancer cells (0.2 to 2 mm in size) in the lymph nodes; (ii) the tumor is 2 cm to 5 cm in size; with metastasis to one to three axillary lymph nodes or to lymph nodes near the breastbone; or (iii) the tumor is larger than 5 cm but has not spread to the axillary lymph nodes. Stage 3A refers to invasive breast cancer in which (i) no tumor is found in the breast or the tumor may be any size, and there is metastasis to four to nine axillary lymph nodes or in the lymph nodes near the breastbone; (ii) the tumor is larger than 5 cm and small groups of breast cancer cells (0.2 mm to 2 mm in size) are found in the lymph nodes; or (iii) the tumor is larger than 5 cm and the cancer has spread to one to three axillary lymph nodes or to the lymph nodes near the breastbone. Stage 3B describes invasive breast cancer in which the tumor is any size and has spread to the chest wall and/or skin of the breast, causing swelling or an ulcer and the tumor has spread (i) to up to nine axillary lymph nodes or (ii) to lymph nodes near the breastbone. Stage 3C refers to invasive breast cancer in which there may be no sign of cancer in the breast or, if there is a tumor, it may be any size and may have spread to the chest wall and/or the skin of the breast and the cancer has spread (i) to ten or more axillary lymph nodes, (ii) to lymph nodes above or below the collarbone, or (iii) to lymph nodes near the breastbone. Stage 4, or advanced, breast cancer refers to invasive breast cancer that has spread beyond the breast and nearby lymph nodes to other organs of the body, such as the lungs, distant lymph nodes, skin, bones, liver, or brain.

In some aspects, the methods disclosed herein treat a stage 0 TNBC. In some aspects, the methods disclosed herein treat a stage 1A TNBC. In some aspects, the methods disclosed herein treat a stage 1B TNBC. In some aspects, the methods disclosed herein treat a stage 2A TNBC. In some aspects, the methods disclosed herein treat a stage 2B TNBC. In some aspects, the methods disclosed herein treat a stage 3A TNBC. In some aspects, the methods disclosed herein treat a stage 3B TNBC. In some aspects, the methods disclosed herein treat a stage 3C TNBC. In some aspects, the methods disclosed herein treat a stage 4 TNBC. In some aspects, the methods disclosed herein treat recurrent TNBC.

### II.E. Glioblastoma

Certain aspects of the present disclosure are directed to methods of treating a glioblastoma in a subject in need thereof comprising administering an extracellular vesicle (EV) comprising an IL-12 moiety. As used herein, glioblastoma, also referred to as “glioblastoma multiforme” or “GBM,” refers to a type of cancer that is a subset of Stage IV astrocytoma. There are several variants of glioblastoma, including giant cell glioblastoma, gliosarcoma, classical glioblastoma, neural glioblastoma, proneural glioblastoma, and mesenchymal glioblastoma. Glioblastoma tumors can be primary (de novo) or secondary.

There is currently no cure for glioblastoma. Standard of care treatment for glioblastoma is surgical resection followed by radiation and chemotherapy (e.g., with temozolomide). Bevacizumab can also be used as a second-line therapy for glioblastoma. The median length of survival after a diagnosis is 15-18 months, while the disease's five-year survival rate is around 10%. Though all glioblastomas recur, initial treatments may keep the tumor controlled for months or even years.

### II.F. Combination Therapies

In some aspects, the methods disclosed herein can comprise administering an EV, e.g., exosome, comprising an IL-12 moiety, in combination with one or more additional therapeutic agents (e.g., immuno-oncology agents). In some aspects, the combination therapy targets multiple elements of the immune pathway. Non-limiting examples of such combinations include: a STING agonist; a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxygenase (IDO), dioxygenase, arginase, or nitric oxide synthetase; a therapy that reverses/prevents T cell anergy or exhaustion; a therapy that triggers an innate immune activation and/or inflammation at a tumor site; administration of immune stimulatory cytokines; or blocking of immuno repressive cytokines. In some aspects, the additional therapeutic agent is an anti-neoplastic agent.

In some aspects, an immuno-oncology agent that can be used in combination with EVs, e.g., exosomes, disclosed herein comprises an immune checkpoint inhibitor (i.e., blocks signaling through the particular immune checkpoint pathway). Non-limiting examples of immune checkpoint inhibitors that can be used in the present methods include a CTLA-4 antagonist (e.g., anti-CTLA-4 antibody), a PD-1 antagonist (e.g., an anti-PD-1 antibody and/or an anti-PD-L1 antibody), a TIM-3 antagonist (e.g., an anti-TIM-3 antibody), a TIGIT antagonist (e.g., an anti-TIGIT antibody), and combinations thereof.

In some aspects, an immuno-oncology agent comprises an immune checkpoint activator (i.e., promotes signaling through the particular immune checkpoint pathway). In certain aspects, the immune checkpoint activator comprises an OX40 agonist (e.g., an anti-OX40 antibody), a LAG-3 agonist (e.g. an anti-LAG-3 antibody), a 4-1BB (CD137) agonist (e.g., an anti-CD137 antibody), a GITR agonist (e.g., an anti-GITR antibody), or any combination thereof.

In some aspects, EVs, e.g., exosomes, disclosed herein can also be used in combination with a STING agonist. Any STING agonist can be used in the methods and compositions disclosed herein. Non-limiting examples of STING agonists include DMXAA, STING agonist-1, ML RR-S2 CDA, ML RR-S2c-di-GMP, ML-RR-S2 cGAMP, 2′3′-c-di-AM(PS)2, 2′3′-cGAMP, 2′3′-cGAMPdFHS, 3′3′-cGAMP, 3′3′-cGAMPdFSH, cAIMP, cAIM(PS)2, 3′3′-cAIMP, 3′3′-cAIMPdFSH, 2′2′-cGAMP, 2′3′-cGAM(PS)2, 3′3′-cGAMP, c-di-AMP, 2′3′-c-di-AMP, 2′3′-c-di-AM(PS)2, c-di-GMP, 2′3′-c-di-GMP, c-di-IMP, c-di-UMP or any combination thereof. In a preferred aspect, the STING agonist is 3′3′-cAIMPdFSH, alternatively named 3-3 cAIMPdFSH. Additional examples of STING agonists that can be used in the compositions and methods disclosed herein include those listed in International Publication No. WO 2021/062060 and/or WO 2019/183578, each of which is incorporated by reference herein in its entirety.

In some aspects, EVs, e.g., exosomes, disclosed herein can also be used in combination with one or more additional immunomodulating agents. Such agents can include, for example, chemotherapy drugs, small molecule drugs, or antibodies that stimulate the immune response to a given cancer. In some aspects, the methods described herein are used in combination with a standard of care treatment (e.g., surgery, radiation, and chemotherapy).

In some aspects, a combination of an EV, e.g., exosome, disclosed herein and a second agent discussed herein (e.g., immune checkpoint inhibitor) can be administered concurrently as a single composition in a pharmaceutically acceptable carrier. In other aspects, a combination of an EV, e.g., exosome, and a second agent discussed herein (e.g., immune checkpoint inhibitor) can be administered concurrently as separate compositions. In further aspects, a combination of an EV, e.g., exosome, and a second agent discussed herein (e.g., immune checkpoint inhibitor) can be administered sequentially. In some aspects, an EV, e.g., exosome, is administered prior to the administration of a second agent (e.g., immune checkpoint inhibitor).

### III. Extracellular Vesicles, e.g., Exosomes

Disclosed herein are EVs, e.g., exosomes, capable of regulating the immune system of a subject. The EVs, e.g., exosomes, useful in the present disclosure have been engineered to comprise an IL-12 moiety.

EVs, e.g., exosomes, described herein are extracellular vesicles with a diameter between about 20-300 nm. In certain aspects, an EV, e.g., exosome, of the present disclosure has a diameter between about 20-290 nm, 20-280 nm, 20-270 nm, 20-260 nm, 20-250 nm, 20-240 nm, 20-230 nm, 20-220 nm, 20-210 nm, 20-200 nm, 20-190 nm, 20-180 nm, 20-170 nm, 20-160 nm, 20-150 nm, 20-140 nm, 20-130 nm, 20-120 nm, 20-110 nm, 20-100 nm, 20-90 nm, 20-80 nm, 20-70 nm, 20-60 nm, 20-50 nm, 20-40 nm, 20-30 nm, 30-300 nm, 30-290 nm, 30-280 nm, 30-270 nm, 30-260 nm, 30-250 nm, 30-240 nm, 30-230 nm, 30-220 nm, 30-210 nm, 30-200 nm, 30-190 nm, 30-180 nm, 30-170 nm, 30-160 nm, 30-150 nm, 30-140 nm, 30-130 nm, 30-120 nm, 30-110 nm, 30-100 nm, 30-90 nm, 30-80 nm, 30-70 nm, 30-60 nm, 30-50 nm, 30-40 nm, 40-300 nm, 40-290 nm, 40-280 nm, 40-270 nm, 40-260 nm, 40-250 nm, 40-240 nm, 40-230 nm, 40-220 nm, 40-210 nm, 40-200 nm, 40-190 nm, 40-180 nm, 40-170 nm, 40-160 nm, 40-150 nm, 40-140 nm, 40-130 nm, 40-120 nm, 40-110 nm, 40-100 nm, 40-90 nm, 40-80 nm, 40-70 nm, 40-60 nm, 40-50 nm, 50-300 nm, 50-290 nm, 50-280 nm, 50-270 nm, 50-260 nm, 50-250 nm, 50-240 nm, 50-230 nm, 50-220 nm, 50-210 nm, 50-200 nm, 50-190 nm, 50-180 nm, 50-170 nm, 50-160 nm, 50-150 nm, 50-140 nm, 50-130 nm, 50-120 nm, 50-110 nm, 50-100 nm, 50-90 nm, 50-80 nm, 50-70 nm, 50-60 nm, 60-300 nm, 60-290 nm, 60-280 nm, 60-270 nm, 60-260 nm, 60-250 nm, 60-240 nm, 60-230 nm, 60-220 nm, 60-210 nm, 60-200 nm, 60-190 nm, 60-180 nm, 60-170 nm, 60-160 nm, 60-150 nm, 60-140 nm, 60-130 nm, 60-120 nm, 60-110 nm, 60-100 nm, 60-90 nm, 60-80 nm, 60-70 nm, 70-300 nm, 70-290 nm, 70-280 nm, 70-270 nm, 70-260 nm, 70-250 nm, 70-240 nm, 70-230 nm, 70-220 nm, 70-210 nm, 70-200 nm, 70-190 nm, 70-180 nm, 70-170 nm, 70-160 nm, 70-150 nm, 70-140 nm, 70-130 nm, 70-120 nm, 70-110 nm, 70-100 nm, 70-90 nm, 70-80 nm, 80-300 nm, 80-290 nm, 80-280 nm, 80-270 nm, 80-260 nm, 80-250 nm, 80-240 nm, 80-230 nm, 80-220 nm, 80-210 nm, 80-200 nm, 80-190 nm, 80-180 nm, 80-170 nm, 80-160 nm, 80-150 nm, 80-140 nm, 80-130 nm, 80-120 nm, 80-110 nm, 80-100 nm, 80-90 nm, 90-300 nm, 90-290 nm, 90-280 nm, 90-270 nm, 90-260 nm, 90-250 nm, 90-240 nm, 90-230 nm, 90-220 nm, 90-210 nm, 90-200 nm, 90-190 nm, 90-180 nm, 90-170 nm, 90-160 nm, 90-150 nm, 90-140 nm, 90-130 nm, 90-120 nm, 90-110 nm, 90-100 nm, 100-300 nm, 110-290 nm, 120-280 nm, 130-270 nm, 140-260 nm, 150-250 nm, 160-240 nm, 170-230 nm, 180-220 nm, or 190-210 nm. The size of the EV, e.g., exosome, described herein can be measured according to methods described, infra.

In some aspects, an EV, e.g., exosome, of the present disclosure comprises a bi-lipid membrane (“EV, e.g., exosome, membrane”), comprising an interior (luminal) surface and an exterior surface. In certain aspects, the interior (luminal) surface faces the inner core (i.e., lumen) of the EV, e.g., exosome. In certain aspects, the exterior surface can be in contact with the endosome, the multivesicular bodies, or the membrane/cytoplasm of a producer cell or a target cell

In some aspects, the EV, e.g., exosome, membrane comprises lipids and fatty acids. In some aspects, the EV, e.g., exosome, membrane comprises phospholipids, glycolipids, fatty acids, sphingolipids, phosphoglycerides, sterols, cholesterols, and phosphatidylserines.

In some aspects, the EV, e.g., exosome, membrane comprises an inner leaflet and an outer leaflet. The composition of the inner and outer leaflet can be determined by transbilayer distribution assays known in the art, see, e.g., Kuypers et al., Biohim Biophys Acta 1985 819:170. In some aspects, the composition of the outer leaflet is between approximately 70-90% choline phospholipids, between approximately 0-15% acidic phospholipids, and between approximately 5-30% phosphatidylethanolamine. In some aspects, the composition of the inner leaflet is between approximately 15-40% choline phospholipids, between approximately 10-50% acidic phospholipids, and between approximately 30-60% phosphatidylethanolamine.

In some aspects, the EV, e.g., exosome, membrane comprises one or more polysaccharide, such as glycan.

In some aspects, the EV, e.g., exosome, of the present disclosure comprises an IL-12 moiety, wherein the IL-12 moiety is linked to the EV via a scaffold moiety, either on the exterior surface of the EV or on the luminal surface of the EV.

In some aspects, the EV, e.g., exosome, of the present disclosure comprises an IL-12 moiety in the lumen of the EV. In other aspects, the EV comprises an IL-12 moiety on the exterior surface of the EV, optionally linked via a first scaffold moiety (e.g., Scaffold X). In other aspects, the EV comprises an IL-12 moiety on the luminal surface of the EV, optionally linked via a scaffold moiety (e.g., Scaffold X or Scaffold Y).

### III.A. Scaffold Moieties

One or more scaffold moieties can be used to anchor an IL-12 moiety to the EV of the present disclosure. In some aspects, the IL-12 moiety is linked to the scaffold moiety. In some aspects, the EV comprises more than one scaffold moiety. In some aspects, the IL-12 is linked to a first scaffold moiety and a second moiety (e.g., a second polypeptide or a polynucleotide) is linked to a second scaffold moiety. In some aspects, the first scaffold moiety and the second scaffold moiety are the same type of scaffold moiety, e.g., the first and second scaffold moieties are both a Scaffold X protein. In some aspects, the first scaffold moiety and the second scaffold moiety are different types of scaffold moiety, e.g., the first scaffold moiety is a Scaffold Y protein and the second scaffold moiety is a Scaffold X protein. In some aspects, the first scaffold moiety is a Scaffold Y, disclosed herein. In some aspects, the first scaffold moiety is a Scaffold X, disclosed herein. In some aspects, the second scaffold moiety is a Scaffold Y, disclosed herein. In some aspects, the second scaffold moiety is a Scaffold X, disclosed herein.

In some aspects, the EV comprises one or more scaffold moieties, which are capable of anchoring, e.g., n IL-12 moiety, to the EV, e.g., exosome, (e.g., either on the luminal surface or on the exterior surface). In certain aspects, the scaffold moiety is a polypeptide (“scaffold protein”). In certain aspects, the scaffold protein comprises an exosome protein or a fragment thereof. In other aspects, scaffold moieties are non-polypeptide moieties. In some aspects, scaffold proteins include various membrane proteins, such as transmembrane proteins, integral proteins and peripheral proteins, enriched on the exosome membranes. They can include various CD proteins, transporters, integrins, lectins, and cadherins. In certain aspects, a scaffold moiety (e.g., scaffold protein) comprises Scaffold X. In other aspects, a scaffold moiety (e.g., exosome protein) comprises Scaffold Y. In further aspects, a scaffold moiety (e.g., exosome protein) comprises both a Scaffold X and a Scaffold Y.

In some aspects, the IL-12 moiety is linked to a scaffold moiety (e.g., Scaffold X) on the exterior surface of the EV. In some aspects, the IL-12 moiety is linked to a scaffold moiety (e.g., Scaffold X) on the luminal surface of the EV. In some aspects, the IL-12 moiety is linked to a scaffold moiety (e.g., Scaffold Y) on the luminal surface of the EV.

### III.A.1. Scaffold X-Engineered EVs, e.g., Exosomes

In some aspects, EVs, e.g., exosomes, of the present disclosure comprise a membrane modified in its composition. For example, their membrane compositions can be modified by changing the protein, lipid, or glycan content of the membrane.

In some aspects, the surface-engineered EVs, e.g., exosomes, are generated by chemical and/or physical methods, such as PEG-induced fusion and/or ultrasonic fusion. In other aspects, the surface-engineered EVs, e.g., exosomes, are generated by genetic engineering. EVs, e.g., exosomes, produced from a genetically-modified producer cell or a progeny of the genetically-modified cell can contain modified membrane compositions. In some aspects, surface-engineered EVs, e.g., exosomes, have scaffold moiety (e.g., exosome protein, e.g., Scaffold X) at a higher or lower density (e.g., higher number) or include a variant or a fragment of the scaffold moiety.

For example, surface (e.g., Scaffold X)-engineered EVs, can be produced from a cell (e.g., HEK293 cells) transformed with an exogenous sequence encoding a scaffold moiety (e.g., exosome proteins, e.g., Scaffold X) or a variant or a fragment thereof. EVs including scaffold moiety expressed from the exogenous sequence can include modified membrane compositions.

Various modifications or fragments of the scaffold moiety can be used for the aspects of the present disclosure. For example, scaffold moiety modified to have enhanced affinity to a binding agent can be used for generating surface-engineered EV that can be purified using the binding agent. Scaffold moieties modified to be more effectively targeted to EVs and/or membranes can be used. Scaffold moieties modified to comprise a minimal fragment required for specific and effective targeting to exosome membranes can be also used.

Scaffold moieties can be engineered to be expressed as a fusion molecule, e.g., fusion molecule of Scaffold X to an IL-12 moiety. For example, the fusion molecule can comprise a scaffold moiety disclosed herein (e.g., Scaffold X, e.g., PTGFRN, BSG, IGSF2, IGSF3, IGSF8, ITGB1, ITGA4, SLC3A2, ATP transporter, or a fragment or a variant thereof) linked to an IL-12 moiety.

In some aspects, the surface (e.g., Scaffold X)-engineered EVs described herein demonstrate superior characteristics compared to EVs known in the art. For example, surface (e.g., Scaffold X)-engineered contain modified proteins more highly enriched on their surface than naturally occurring EVs or the EVs produced using conventional exosome proteins. Moreover, the surface (e.g., Scaffold X)-engineered EVs of the present disclosure can have greater, more specific, or more controlled biological activity compared to naturally occurring EVs or the EVs produced using conventional exosome proteins.

In some aspects, the Scaffold X comprises Prostaglandin F2 receptor negative regulator (the PTGFRN polypeptide). The PTGFRN protein can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full length amino acid sequence of the human PTGFRN protein (Uniprot Accession No. Q9P2B2) is shown at Table 2 as SEQ ID NO: 1. The PTGFRN polypeptide contains a signal peptide (amino acids 1 to 25 of SEQ ID NO: 1), the extracellular domain (amino acids 26 to 832 of SEQ ID NO: 1), a transmembrane domain (amino acids 833 to 853 of SEQ ID NO: 1), and a cytoplasmic domain (amino acids 854 to 879 of SEQ ID NO: 1). The mature PTGFRN polypeptide consists of SEQ ID NO: 1 without the signal peptide, i.e., amino acids 26 to 879 of SEQ ID NO: 1. In some aspects, a PTGFRN polypeptide fragment useful for the present disclosure comprises a transmembrane domain of the PTGFRN polypeptide. In other aspects, a PTGFRN polypeptide fragment useful for the present disclosure comprises the transmembrane domain of the PTGFRN polypeptide and (i) at least five, at least 10, at least 15, at least 20, at least 25, at least 30, at least 40, at least 50, at least 70, at least 80, at least 90, at least 1%, at least 110, at least 120, at least 130, at least 140, at least 150 amino acids at the N terminus of the transmembrane domain, (ii) at least five, at least 10, at least 15, at least 20, or at least 25 amino acids at the C terminus of the transmembrane domain, or both (i) and (ii).

In some aspects, the fragments of PTGFRN polypeptide lack one or more functional or structural domains, such as IgV.

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 1. In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 1. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 33. In other aspects, the Scaffold X comprises the amino acid sequence of SEQ ID NO: 33, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 33 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 33.

In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 9, 14, 21, 22, or 23. In other aspects, the Scaffold X comprises the amino acid sequence of SEQ ID NO: 9, 14, 21, 22, or 23, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 9, 14, 21, 22, or 23 and one amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 9, 14, 21, 22, or 23.

TABLE 2
Exemplary Scaffolf X Protein Sequences.

| Protein | Sequence |
|---|---|
| The | MGRLASRPLLLALLSLALCRGRVVRVPTATLVRVVGTELVIPCNVSDYDGPSEQNFD |
| PTGFRN | WSF |
| Protein | SSLGSSFVELASTWEVGFPAQLYQERLQRGEILLRRTANDAVELHIKNVQPSDQGHY |
| (SEQ ID | KCS |
| NO: 1) | TPSTDATVQGNYEDTVQVKVLADSLHVGPSARPPPSLSLREGEPFELRCTAASASPL |
| | HTH |
| | LALLWEVHRGPARRSVLALTHEGRFHPGLGYEQRYHSGDVRLDTVGSDAYRLSVSRA |
| | LSA |
| | DQGSYRCIVSEWIAEQGNWQEIQEKAVEVATVVIQPSVLRAAVPKNVSVAEGKELDL |
| | TCN |
| | ITTDRADDVRPEVTWSFSRMPDSTLPGSRVLARLDRDSLVHSSPHVALSHVDARSYH |
| | LLV |
| | RDVSKENSGYYYCHVSLWAPGHNRSWHKVAEAVSSPAGVGVTWLEPDYQVYLNASKV |
| | PGF |
| | ADDPTELACRVVDTKSGEANVRFTVSWYYRMNRRSDNVVTSELLAVMDGDWTLKYGE |
| | RSK |
| | QRAQDGDFIFSKEHTDTFNFRIQRTTEEDRGNYYCVVSAWTKORNNSWVKSKDVFSK |
| | PVN |
| | IFWALEDSVLVVKARQPKPFFAAGNTFEMTCKVSSKNIKSPRYSVLIMAEKPVGDLS |
| | SPN |
| | ETKYIISLDQDSVVKLENWTDASRVDGVVLEKVQEDEFRYRMYQTQVSDAGLYRCMV |
| | TAW |
| | SPVRGSLWREAATSLSNPIEIDFQTSGPIFNASVHSDTPSVIRGDLIKLFCIITVEG |
| | AAL |
| | DPDDMAFDVSWFAVHSFGLDKAPVLLSSLDRKGIVTTSRRDWKSDLSLERVSVLEFL |
| | LQV |
| | HGSEDQDFGNYYCSVTPWVKSPTGSWQKEAEIHSKPVFITVKMDVLNAFKYPLLIGV |
| | GLS |
| | TVIGLLSCLIGYCSSHWCCKKEVQETRRERRRLMSMEMD |
| The | GPIFNASVHSDTPSVIRGDLIKLFCIITVEGAALDPDDMAFDVSWFAVHSFGLDKAP |
| PTGFRN | VLL |
| protein | SSLDRKGIVTTSRRDWKSDLSLERVSVLEFLLQVHGSEDQDFGNYYCSVTPWVKSPT |
| Fragment | GSW |
| (SEQ ID | QKEAEIHSKPVFITVKMDVLNAFKYPLLIGVGLSTVIGLLSCLIGYCSSHWCCKKEV |
| NO: 33) | QET |
| | RRERRRLMSMEM |
| | 687-878 of SEQ ID NO: 1 |
| The BSG | MAAALFVLLG FALLGTHGAS GAAGFVQAPL SQQRWVGGSV ELHCEAVGSP |
| protein | VPEIQWWFEG QGPNDTCSQL WDGARLDRVH IHATYHQHAA STISIDTLVE |
| (SEQ ID | EDTGTYECRA SNDPDRNHLT RAPRVKWVRA QAVVLVLEPG TVFTTVEDLG |
| NO: 9) | SKILLTCSLN DSATEVTGHR WLKGGVVLKE DALPGQKTEF KVDSDDQWGE |
| | YSCVFLPEPM GTANIQLHGP PRVKAVKSSE HINEGETAML VCKSESVPPV |
| | TDWAWYKITD SEDKALMNGS ESRFFVSSSQ GRSELHIENL NMEADPGQYR |
| | CNGTSSKGSD QAIITLRVRS HLAALWPFLG IVAEVLVLVT IIFIYEKRRK |
| | PEDVLDDDDA GSAPLKSSGQ HONDKGKNVR QRNSS |
| The IGSF8 | MGALRPTLLP PSLPLLLLLM LGMGCWAREV LVPEGPLYRV AGTAVSISCN |
| protein | VTGYEGPAQQ NFEWFLYRPE APDTALGIVS TKDTQFSYAV FKSRVVAGEV |
| (SEQ ID | QVQRLQGDAV VLKIARLQAQ DAGIYECHTP STDTRYLGSY SGKVELRVLP |
| NO: 14) | DVLQVSAAPP GPRGRQAPTS PPRMTVHEGQ ELALGCLART STQKHTHLAV |
| | SFGRSVPEAP VGRSTLQEVV GIRSDLAVEA GAPYAERLAA GELRLGKEGT |
| | DRYRMVVGGA QAGDAGTYHC TAAEWIQDPD GSWAQIAEKR AVLAHVDVQT |
| | LSSQLAVTVG PGERRIGPGE PLELLCNVSG ALPPAGRHAA YSVGWEMAPA |
| | GAPGPGRLVA QLDTEGVGSL GPGYEGRHIA MEKVASRTYR LRLEAARPGD |
| | AGTYRCLAKA YVRGSGTRLR EAASARSRPL PVHVREEGVV LEAVAWLAGG |
| | TVYRGETASL LCNISVRGGP PGLRLAASWW VERPEDGELS SVPAQLVGGV |
| | GQDGVAELGV RPGGGPVSVE LVGPRSHRLR LHSLGPEDEG VYHCAPSAWV |
| | QHADYSWYQA GSARSGPVTV YPYMHALDTL FVPLLVGTGV ALVTGATVLG |
| | TITCCFMKRL RKR |
| The ITGB1 | MNLQPIFWIG LISSVCCVFA QTDENRCLKA NAKSCGECIQ AGPNCGWCTN |
| protein | STFLQEGMPT SARCDDLEAL KKKGCPPDDI ENPRGSKDIK KNKNVTNRSK |
| (SEQ ID | GTAEKLKPED ITQIQPQQLV LRLRSGEPQT FTLKFKRAED YPIDLYYLMD |
| NO: 21) | LSYSMKDDLE NVKSLGTDLM NEMRRITSDF RIGFGSFVEK TVMPYISTTP |
| | AKLRNPCTSE QNCTSPFSYK NVLSLTNKGE VFNELVGKQR ISGNLDSPEG |
| | GFDAIMQVAV CGSLIGWRNV TRLLVFSTDA GFHFAGDGKL GGIVLPNDGQ |
| | CHLENNMYTM SHYYDYPSIA HLVQKLSENN IQTIFAVTEE FQPVYKELKN |
| | LIPKSAVGTL SANSSNVIQL IIDAYNSLSS EVILENGKLS EGVTISYKSY |
| | CKNGVNGTGE NGRKCSNISI GDEVQFEISI TSNKCPKKDS DSFKIRPLFE |
| | TEEVEVILQY ICECECQSEG IPESPKCHEG NGTFECGACR CNEGRVGRHC |
| | ECSTDEVNSE DMDAYCRKEN SSEICSNNGE CVCGQCVCRK RDNTNEIYSG |
| | ASNGQICNGR GICECGVCKC TDPKFQGQTC EMCQTCLGVC AEHKECVQCR |
| | AFNKGEKKDT CTQECSYFNI TKVESRDKLP QPVQPDPVSH CKEKDVDDCW |
| | FYFTYSVNGN NEVMVHVVEN PECPTGPDII PIVAGVVAGI VLIGLALLLI |
| | WKLLMIIHDR REFAKFEKEK MNAKWDTGEN PIYKSAVTTV VNPKYEGK |
| The ITGA4 | MAWEARREPG PRRAAVRETV MLLLCLGVPT GRPYNVDTES ALLYQGPHNT |
| protein | LFGYSVVLHS HGANRWLLVG APTANWLANA SVINPGAIYR CRIGKNPGQT |
| (SEQ ID | CEQLQLGSPN GEPCGKTCLE ERDNQWLGVT LSRQPGENGS IVTCGHRWKN |
| NO: 22) | IFYIKNENKL PTGGCYGVPP DLRTELSKRI APCYQDYVKK FGENFASCQA |
| | GISSFYTKDL IVMGAPGSSY WTGSLFVYNI TINKYKAFLD KQNQVKFGSY |
| | LGYSVGAGHF RSQHTTEVVG GAPQHEQIGK AYIFSIDEKE LNILHEMKGK |
| | KLGSYFGASV CAVDLNADGF SDLLVGAPMQ STIREEGRVF VYINSGSGAV |
| | MNAMETNLVG SDKYAARFGE SIVNLGDIDN DGFEDVAIGA PQEDDLQGAI |
| | YIYNGRADGI SSTESQRIEG LQISKSLSMF GQSISGQIDA DNNGYVDVAV |
| | GAFRSDSAVL LRTRPVVIVD ASLSHPESVN RTKFDCVENG WPSVCIDLTL |
| | CFSYKGKEVP GYIVLFYNMS LDVNRKAESP PRFYFSSNGT SDVITGSIQV |
| | SSREANCRTH QAFMRKDVRD ILTPIQIEAA YHLGPHVISK RSTEEFPPLQ |
| | PILQQKKEKD IMKKTINFAR FCAHENCSAD LOVSAKIGFL KPHENKTYLA |
| | VGSMKTLMLN VSLFNAGDDA YETTLHVKLP VGLYFIKILE LEEKQINCEV |
| | TDNSGVVQLD CSIGYIYVDH LSRIDISFLL DVSSLSRAEE DLSITVHATC |
| | ENEEEMDNLK HSRVTVAIPL KYEVKLTVHG FVNPTSFVYG SNDENEPETC |
| | MVEKMNLTFH VINTGNSMAP NVSVEIMVPN SFSPQTDKLF NILDVQTTTG |
| | ECHFENYQRV CALEQQKSAM QTLKGIVRFL SKTDKRLLYC IKADPHCLNF |
| | LCNFGKMESG KEASVHIQLE GRPSILEMDE TSALKFEIRA TGFPEPNPRV |
| | IELNKDENVA HVLLEGLHHQ RPKRYFTIVI ISSSLLLGLI VLLLISYVMW |
| | KAGFFKRQYK SILQEENRRD SWSYINSKSN DD |
| The | MELQPPEASI AVVSIPROLP GSHSEAGVQG LSAGDDSELG SHCVAQTGLE |
| SLC3A2 | LLASGDPLPS ASQNAEMIET GSDCVTQAGL QLLASSDPPA LASKNAEVTG |
| Protein, | TMSQDTEVDM KEVELNELEP EKQPMNAASG AAMSLAGAEK NGLVKIKVAE |
| where | DEAEAAAAAK FTGLSKEELL KVAGSPGWVR TRWALLLLFW LGWLGMLAGA |
| the first | VVIIVRAPRC RELPAQKWWH TGALYRIGDL QAFQGHGAGN LAGLKGRLDY |
| Met is | LSSLKVKGLV LGPIHKNQKD DVAQTDLLQI DPNFGSKEDF DSLLQSAKKK |
| processed, | SIRVILDLTP NYRGENSWFS TQVDTVATKV KDALEFWLQA GVDGFQVRDI |
| (SEQ ID | ENLKDASSFL AEWQNITKGF SEDRLLIAGT NSSDLQQILS LLESNKDLLL |
| NO: 23) | TSSYLSDSGS TGEHTKSLVT QYLNATGNRW CSWSLSQARL LTSFLPAQLL |
| | RLYQLMLFTL PGTPVFSYGD EIGLDAAALP GQPMEAPVML WDESSFPDIP |
| | GAVSANMTVK GOSEDPGSLL SLFRRLSDQR SKERSLLHGD FHAFSAGPGL |
| | FSYIRHWDQN ERFLVVLNFG DVGLSAGLQA SDLPASASLP AKADLLLSTQ |
| | PGREEGSPLE LERLKLEPHE GLLLRFPYAA |

In some aspects, a Scaffold X comprises Basigin (the BSG protein), represented by SEQ ID NO: 9. The BSG protein is also known as 5F7, Collagenase stimulatory factor, Extracellular matrix metalloproteinase inducer (EMMPRIN), Leukocyte activation antigen M6, OK blood group antigen, Tumor cell-derived collagenase stimulatory factor (TCSF), or CD147. The Uniprot number for the human BSG protein is P35613. The signal peptide of the BSG protein is amino acid 1 to 21 of SEQ ID NO: 9. Amino acids 138-323 of SEQ ID NO: 9 is the extracellular domain, amino acids 324 to 344 is the transmembrane domain, and amino acids 345 to 385 of SEQ ID NO: 9 is the cytoplasmic domain.

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 22 to 385 of SEQ ID NO: 9. In some aspects, the fragments of BSG polypeptide lack one or more functional or structural domains, such as IgV, e.g., amino acids 221 to 315 of SEQ ID NO: 9. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 10, 11, or 12. In other aspects, the Scaffold X comprises the amino acid sequence of SEQ ID NO: 10, 11, or 12, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 10, 11, or 12 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 10, 11, or 12.

In some aspects, a Scaffold X comprises Immunoglobulin superfamily member 8 (IgSF8 or the IGSF8 protein), which is also known as CD81 partner 3, Glu-Trp-Ile EWI motif-containing protein 2 (EWI-2), Keratinocytes-associated transmembrane protein 4 (KCT-4), LIR-D1, Prostaglandin regulatory-like protein (PGRL) or CD316. The full length human IGSF8 protein is accession no. Q969P0 in Uniprot and is shown as SEQ ID NO: 14 herein. The human IGSF8 protein has a signal peptide (amino acids 1 to 27 of SEQ ID NO: 14), an extracellular domain (amino acids 28 to 579 of SEQ ID NO: 14), a transmembrane domain (amino acids 580 to 600 of SEQ ID NO: 14), and a cytoplasmic domain (amino acids 601 to 613 of SEQ ID NO: 14).

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 28 to 613 of SEQ ID NO: 14. In some aspects, the IGSF8 protein lack one or more functional or structural domains, such as IgV. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 15, 16, 17, or 18. In other aspects, the Scaffold X comprises the amino acid sequence of SEQ ID NO: 15, 16, 17, or 18, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID 15, 16, 17, or 18 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 15, 16, 17, or 18.

In some aspects, a Scaffold X for the present disclosure comprises Immunoglobulin superfamily member 3 (IgSF3 or the IGSF3 protein), which is also known as Glu-Trp-Ile EWI motif-containing protein 3 (EWI-3), and is shown as the amino acid sequence of SEQ ID NO: 20. The human IGSF3 protein has a signal peptide (amino acids 1 to 19 of SEQ ID NO: 20), an extracellular domain (amino acids 20 to 1124 of SEQ ID NO: 20), a transmembrane domain (amino acids 1125 to 1145 of SEQ ID NO: 20), and a cytoplasmic domain (amino acids 1146 to 1194 of SEQ ID NO: 20).

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 28 to 613 of SEQ ID NO: 20. In some aspects, the IGSF3 protein lack one or more functional or structural domains, such as IgV.

In some aspects, a Scaffold X for the present disclosure comprises Integrin beta-1 (the ITGB1 protein), which is also known as Fibronectin receptor subunit beta, Glycoprotein IIa (GPIIA), VLA-4 subunit beta, or CD29, and is shown as the amino acid sequence of SEQ ID NO: 21. The human ITGB1 protein has a signal peptide (amino acids 1 to 20 of SEQ ID NO: 21), an extracellular domain (amino acids 21 to 728 of SEQ ID NO: 21), a transmembrane domain (amino acids 729 to 751 of SEQ ID NO: 21), and a cytoplasmic domain (amino acids 752 to 798 of SEQ ID NO: 21).

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 21 to 798 of SEQ ID NO: 21. In some aspects, the ITGB1 protein lack one or more functional or structural domains, such as IgV.

In other aspects, the Scaffold X comprises the ITGA4 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 22 without the signal peptide (amino acids 1 to 33 of SEQ ID NO: 22). In some aspects, the ITGA4 protein lacks one or more functional or structural domains, such as IgV.

In other aspects, the Scaffold X comprises the SLC3A2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 23 without the signal peptide. In some aspects, the SLC3A2 protein lacks one or more functional or structural domains, such as IgV.

Non-limiting examples of other Scaffold X proteins can be found at US Patent No. U.S. Ser. No. 10/195,290B1, issued Feb. 5, 2019, which is incorporated by reference in its entireties.

In some aspects, the sequence encodes a fragment of the scaffold moiety lacking at least 5, 10, 50, 100, 200, 300, 400, 500, 600, 700, or 800 amino acids from the N-terminus of the native protein. In some aspects, the sequence encodes a fragment of the scaffold moiety lacking at least 5, 10, 50, 100, 200, 300, 400, 500, 600, 700, or 800 amino acids from the C-terminus of the native protein. In some aspects, the sequence encodes a fragment of the scaffold moiety lacking at least 5, 10, 50, 100, 200, 300, 400, 500, 600, 700, or 800 amino acids from both the N-terminus and C-terminus of the native protein. In some aspects, the sequence encodes a fragment of the scaffold moiety lacking one or more functional or structural domains of the native protein.

In some aspects, the scaffold moieties, e.g., Scaffold X, e.g., a PTGFRN protein, are linked to one or more heterologous proteins. The one or more heterologous proteins can be linked to the N-terminus of the scaffold moieties. The one or more heterologous proteins can be linked to the C-terminus of the scaffold moieties. In some aspects, the one or more heterologous proteins are linked to both the N-terminus and the C-terminus of the scaffold moieties. In some aspects, the heterologous protein is a mammalian protein. In some aspects, the heterologous protein is a human protein.

In some aspects, Scaffold X can be used to link any moiety, e.g., an IL-12 moiety, to the luminal surface and on the exterior surface of the EV, e.g., exosome, at the same time. For example, the PTGFRN polypeptide can be used to link an IL-12 moiety inside the lumen (e.g., on the luminal surface) in addition to the exterior surface of the EV, e.g., exosome. In some aspects, Scaffold X is a scaffold protein that is capable of anchoring the IL-12 on the luminal surface of the EV and/or on the exterior surface of the EV.

### III.A.2. Scaffold Y-Engineered EVs, e.g., Exosomes

In some aspects, EVs, e.g., exosomes, of the present disclosure comprise an internal space (i.e., lumen) that is different from that of the naturally occurring EVs. For example, the EV can be changed such that the composition in the luminal surface of the EV, e.g., exosome has the protein, lipid, or glycan content different from that of the naturally-occurring exosomes.

In some aspects, engineered EVs, e.g., exosomes, can be produced from a cell transformed with an exogenous sequence encoding a scaffold moiety (e.g., exosome proteins, e.g., Scaffold Y) or a modification or a fragment of the scaffold moiety that changes the composition or content of the luminal surface of the EV, e.g., exosome. Various modifications or fragments of the exosome protein that can be expressed on the luminal surface of the EV, e.g., exosome, can be used for the aspects of the present disclosure.

In some aspects, the exosome proteins that can change the luminal surface of the EVs, e.g., exosomes, include, but are not limited to, the myristoylated alanine rich Protein Kinase C substrate (MARCKS) protein, the myristoylated alanine rich Protein Kinase C substrate like 1 (MARCKSL1) protein, the brain acid soluble protein 1 (BASP1) protein, or any combination thereof.

In some aspects, Scaffold Y comprises the MARCKS protein (Uniprot accession no. P29966). The MARCKS protein is also known as protein kinase C substrate, 80 kDa protein, light chain. The full-length human MARCKS protein is 332 amino acids in length and comprises a calmodulin-binding domain at amino acid residues 152-176. In some aspects, Scaffold Y comprises the MARCKSL1 protein (Uniprot accession no. P49006). The MARCKSL1 protein is also known as MARCKS-like protein 1, and macrophage myristoylated alanine-rich C kinase substrate. The full-length human MARCKSL1 protein is 195 amino acids in length. The MARCKSL1 protein has an effector domain involved in lipid-binding and calmodulin-binding at amino acid residues 87-110. In some aspects, the Scaffold Y comprises the BASP1 protein (Uniprot accession number P80723). The BASP1 protein is also known as 22 kDa neuronal tissue-enriched acidic protein or neuronal axonal membrane protein NAP-22. The full-length human BASP1 protein sequence (isomer 1) is 227 amino acids in length. An isomer produced by an alternative splicing is missing amino acids 88 to 141 from SEQ ID NO: 49 (isomer 1). Table 3 provides the full-length sequences for the exemplary Scaffold Y disclosed herein (i.e., the MARCKS, MARCKSL1, and BASP1 proteins).

TABLE 3
Exemplary Scaffold Y Protein Sequences.

| | |
|---|---|
| Protein | Sequence |
| The MARCKS | MGAQFSKTAA KGEAAAERPG EAAVASSPSK ANGQENGHVK VNGDASPAAA |
| protein | ESGAKEELQA NGSAPAADKE EPAAAGSGAA SPSAAEKGEP AAAAAPEAGA |
| (SEQ ID | SPVEKEAPAE GEAAEPGSPT AAEGEAASAA SSTSSPKAED GATPSPSNET |
| NO: 47) | PKKKKKRFSF KKSFKLSGFS FKKNKKEAGE GGEAEAPAAE GGKDEAAGGA |
| | AAAAAEAGAA SGEQAAAPGE EAAAGEEGAA GGDPQEAKPQ EAAVAPEKPP |
| | ASDETKAAEE PSKVEEKKAE EAGASAAACE APSAAGPGAP PEQEAAPAEE |
| | PAAAAASSAC AAPSQEAQPE CSPEAPPAEA AE |
| The | MGSQSSKAPR GDVTAEEAAG ASPAKANGQE NGHVKSNGDL SPKGEGESPP |
| MARCKSL1 | VNGTDEAAGA TGDAIEPAPP SQGAEAKGEV PPKETPKKKK KFSFKKPEKL |
| protein | SGLSFKRNRK EGGGDSSASS PTEEEQEQGE IGACSDEGTA QEGKAAATPE |
| (SEQ ID | SQEPQAKGAE ASAASEEEAG PQATEPSTPS GPESGPTPAS AEQNE |
| NO: 48) | |
| The BASP1 | MGGKLSKKKK GYNVNDEKAK EKDKKAEGAA TEEEGTPKES EPQAAAEPAE |
| protein | AKEGKEKPDQ DAEGKAEEKE GEKDAAAAKE EAPKAEPEKT EGAAEAKAEP |
| (SEQ ID | PKAPEQEQAA PGPAAGGEAP KAAEAAAAPA ESAAPAAGEE PSKEEGEPKK |
| NO: 49) | TEAPAAPAAQ ETKSDGAPAS DSKPGSSEAA PSSKETPAAT EAPSSTPKAQ |
| | GPAASAEEPK PVEAPAANSD QTVTVKE |

The mature BASP1 protein sequence is missing the first Met from SEQ TD NO: 49 and thus contains amino acids 2 to 227 of SEQ TD NO: 49. Similarly, the mature MARCKS and MARCKSL1 proteins also lack the first Met from SEQ TD NOs: 47 and 48, respectively. Accordingly, the mature MARCKS protein contains amino acids 2 to 332 of SEQ ID NO: 47. The mature MARCKSL1 protein contains amino acids 2 to 227 of SEQ ID NO: 48.

In other aspects, Scaffold Y useful for the present disclosure comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 2 to 227 of SEQ ID NO: 49. In other aspects, a Scaffold Y useful for the present disclosure comprises the amino acid sequence of SEQ ID NO: 49, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof.

In some aspects, the protein sequence of any of SEQ ID NOs: 47-49 without Met at amino acid residue 1 of the SEQ ID NOs: 47-49 is sufficient to be a Scaffold Y for the present disclosure (e.g., scaffold moiety linked to an IL-12 moiety. In some aspects, the Scaffold Y comprises an amino acid sequence at least about 70%, at least about 75%, at least about 80%, at least about 85%, at least about 90%, at least about 95%, at least about 96%, at least about 97%, at least about 98%, at least about 99%, or about 100% identical to any one of SEQ ID NO: 47-49 without Met at amino acid residue 1 of the SEQ ID NOs: 47-49.

Scaffold Y-engineered EVs, e.g., exosomes described herein can be produced from a cell transformed with a sequence set forth in SEQ ID NOs: 47-49 without Met at amino acid residue 1 of the SEQ ID NOs: 47-49.

In some aspects, the Scaffold Y protein useful for the present disclosure comprises an “N-terminus domain” (ND) and an “effector domain” (ED), wherein the ND and/or the ED are associated with the luminal surface of the EV, e.g., an exosome. In some aspects, the Scaffold Y protein useful for the present disclosure comprises an intracellular domain, a transmembrane domain, and an extracellular domain; wherein the intracellular domain comprises an “N-terminus domain” (ND) and an “effector domain” (ED), wherein the ND and/or the ED are associated with the luminal surface of the EV, e.g., an exosome. As used herein the term “associated with” refers to the interaction between a scaffold protein with the luminal surface of the EV, e.g., and exosome, that does not involve covalent linking to a membrane component. For example, the scaffolds useful for the present disclosure can be associated with the luminal surface of the EV, e.g., via a lipid anchor (e.g., myristic acid), and/or a polybasic domain that interacts electrostatically with the negatively charged head of membrane phospholipids. In other aspects, the Scaffold Y protein comprises an N-terminus domain (ND) and an effector domain (ED), wherein the ND is associated with the luminal surface of the EV and the ED are associated with the luminal surface of the EV by an ionic interaction, wherein the ED comprises at least two, at least three, at least four, at least five, at least six, or at least seven contiguous basic amino acids, e.g., lysines (Lys), in sequence.

In other aspects, the Scaffold Y protein comprises an N-terminus domain (ND) and an effector domain (ED), wherein the ND is associated with the luminal surface of the EV, e.g., exosome, and the ED is associated with the luminal surface of the EV by an ionic interaction, wherein the ED comprises at least two, at least three, at least four, at least five, at least six, or at least seven contiguous basic amino acids, e.g., lysines (Lys), in sequence.

In some aspects, the ND is associated with the luminal surface of the EV, e.g., an exosome, via lipidation, e.g., via myristoylation. In some aspects, the ND has Gly at the N terminus. In some aspects, the N-terminal Gly is myristoylated.

In some aspects, the ED is associated with the luminal surface of the EV, e.g., an exosome, by an ionic interaction. In some aspects, the ED is associated with the luminal surface of the EV, e.g., an exosome, by an electrostatic interaction, in particular, an attractive electrostatic interaction.

In some aspects, the ED comprises (i) a basic amino acid (e.g., lysine), or (ii) two or more basic amino acids (e.g., lysine) next to each other in a polypeptide sequence. In some aspects, the basic amino acid is lysine (Lys; K), arginine (Arg, R), or Histidine (His, H). In some aspects, the basic amino acid is (Lys)ₙ, wherein n is an integer between 1 and 10.

In other aspects, the ED comprises at least a lysine and the ND comprises a lysine at the C terminus if the N terminus of the ED is directly linked to lysine at the C terminus of the ND, i.e., the lysine is in the N terminus of the ED and is fused to the lysine in the C terminus of the ND. In other aspects, the ED comprises at least two lysines, at least three lysines, at least four lysines, at least five lysines, at least six lysines, or at least seven lysines when the N terminus of the ED is linked to the C terminus of the ND by a linker, e.g., one or more amino acids.

Non-limiting examples of the Scaffold Y protein useful for the present disclosure are disclosed in International Publication No. WO/2019/099942, which is incorporated by reference herein in its entirety.

In some aspects, the Scaffold Y protein useful for the present disclosure does not contain an N-terminal Met. In some aspects, the Scaffold Y protein comprises a lipidated amino acid, e.g., a myristoylated amino acid, at the N-terminus of the scaffold protein, which functions as a lipid anchor. In some aspects, the amino acid residue at the N-terminus of the scaffold protein is Gly. The presence of an N-terminal Gly is an absolute requirement for N-myristoylation. In some aspects, the amino acid residue at the N-terminus of the scaffold protein is synthetic. In some aspects, the amino acid residue at the N-terminus of the scaffold protein is a glycine analog, e.g., allylglycine, butylglycine, or propargylglycine.

In other aspects, the lipid anchor can be any lipid anchor known in the art, e.g., palmitic acid or glycosylphosphatidylinositols. Under unusual circumstances, e.g., by using a culture medium where myristic acid is limiting, some other fatty acids including shorter-chain and unsaturated, can be attached to the N-terminal glycine. For example, in BK channels, myristate has been reported to be attached post-translationally to internal serine/threonine or tyrosine residues via a hydroxyester linkage. Membrane anchors known in the art are presented in the following table:

| Modification | Modifying Group |
|---|---|
| S-Palmitoylation | 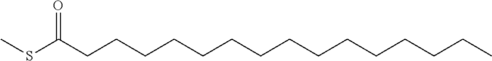 [SMILES: CCCCCCCCCCCCCCCC(=O)SC] |
| N-Palmitoylation | 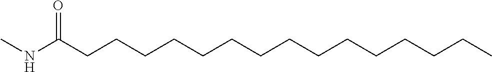 [SMILES: CCCCCCCCCCCCCCCC(=O)NC] |
| N-Myristoylation | 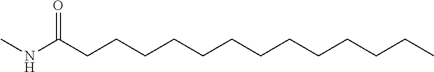 [SMILES: CCCCCCCCCCCCCC(=O)NC] |
| O-Acylation | 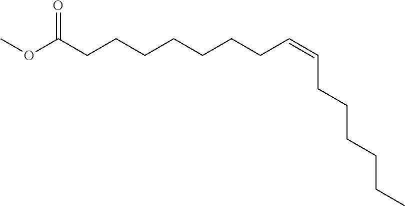 [SMILES: CCCCCC/C=C\CCCCCCCC(=O)OC] |
| Farnesylation | 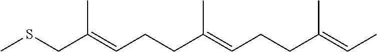 [SMILES: C/C=C(\C)CC/C=C(\C)CC/C=C(\C)CSC] |
| Geranylgeranylation | 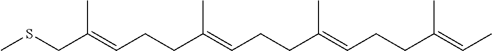 [SMILES: C/C=C(\C)CC/C=C(\C)CC/C=C(\C)CC/C=C(\C)CSC] |
| Cholesterol | 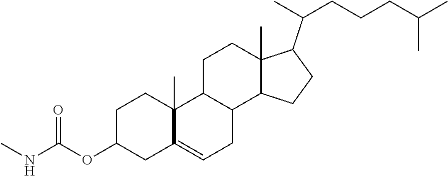 |

### III.B. Linkers

As described supra, extracellular vesicles (EVs) of the present disclosure (e.g., exosomes and nanovesicles) can comprises one or more linkers that link a molecule of interest (e.g., an IL-12 moiety) to the EVs (e.g., to the exterior surface or on the luminal surface). In some aspects, an IL-12 moiety is linked to the EVs directly or via a scaffold moiety (e.g., Scaffold X or Scaffold Y). In certain aspects, the IL-12 moiety is linked to the scaffold moiety by a linker. In certain aspects, the IL-12 moiety is linked to the second scaffold moiety by a linker.

In certain aspects, the IL-12 moiety is linked to the exterior surface of an exosome via Scaffold X. In further aspects, the IL-12 moiety is linked to the luminal surface of an exosome via Scaffold X or Scaffold Y. The linker can be any chemical moiety known in the art.

In some aspects, two or more linkers can be linked in tandem. When multiple linkers are present, each of the linkers can be the same or different. Generally, linkers provide flexibility or prevent/ameliorate steric hindrances. Linkers are not typically cleaved; however, in certain aspects, such cleavage can be desirable. Accordingly, in some aspects, a linker can comprise one or more protease-cleavable sites, which can be located within the sequence of the linker or flanking the linker at either end of the linker sequence.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

In some aspects, the linker is a non-polypeptide moiety.

Linkers can be susceptible to cleavage (“cleavable linker”) thereby facilitating release of the biologically active molecule (e.g., An IL-12 moiety).

In some aspects, the linker is a “reduction-sensitive linker.” In some aspects, the reduction-sensitive linker contains a disulfide bond. In some aspects, the linker is an “acid labile linker.” In some aspects, the acid labile linker contains hydrazone. Suitable acid labile linkers also include, for example, a cis-aconitic linker, a hydrazide linker, a thiocarbamoyl linker, or any combination thereof.

In some aspects, the linker comprises a non-cleavable linker.

### IV. Producer Cell for Production of Engineered Exosomes

EVs, e.g., exosomes, of the present disclosure can be produced from a cell grown in vitro or a body fluid of a subject. When exosomes are produced from in vitro cell culture, various producer cells, e.g., HEK293 cells, CHO cells, and MSCs, can be used. In certain aspects, a producer cell is not a dendritic cell, macrophage, B cell, mast cell, neutrophil, Kupffer-Browicz cell, cell derived from any of these cells, or any combination thereof.

The producer cell can be genetically modified to comprise exogenous sequences encoding An IL-12 moiety to produce EVs described herein. The genetically-modified producer cell can contain the exogenous sequence by transient or stable transformation. The exogenous sequence can be transformed as a plasmid. In some aspects, the exogenous sequence is a vector. The exogenous sequences can be stably integrated into a genomic sequence of the producer cell, at a targeted site or in a random site. In some aspects, a stable cell line is generated for production of lumen-engineered exosomes.

The exogenous sequences can be inserted into a genomic sequence of the producer cell, located within, upstream (5′-end) or downstream (3′-end) of an endogenous sequence encoding an exosome protein. Various methods known in the art can be used for the introduction of the exogenous sequences into the producer cell. For example, cells modified using various gene editing methods (e.g., methods using a homologous recombination, transposon-mediated system, loxP-Cre system, CRISPR/Cas9 or TALEN) are within the scope of the present disclosure.

The exogenous sequences can comprise a sequence encoding a scaffold moiety disclosed herein or a fragment or variant thereof. An extra copy of the sequence encoding a scaffold moiety can be introduced to produce an exosome described herein (e.g., having a higher density of a scaffold moiety on the surface or on the luminal surface of the EV, e.g., exosome). An exogenous sequence encoding a modification or a fragment of a scaffold moiety can be introduced to produce a lumen-engineered and/or surface-engineered exosome containing the modification or the fragment of the scaffold moiety.

In some aspects, a producer cell can be modified, e.g., transfected, with one or more vectors encoding a scaffold moiety linked to an IL-12 moiety.

In some aspects, a producer cell disclosed herein is further modified to comprise an additional exogenous sequence. For example, an additional exogenous sequence can be introduced to modulate endogenous gene expression, or produce an exosome including a certain polypeptide as a payload (e.g., an IL-12 moiety). In some aspects, the producer cell is modified to comprise two exogenous sequences, one encoding a scaffold moiety (e.g., Scaffold X and/or Scaffold Y), or a variant or a fragment thereof, and the other encoding a payload e.g., an IL-12 moiety). In some aspects, the producer cell is modified to comprise two exogenous sequences, one encoding a scaffold moiety disclosed herein, or a variant or a fragment thereof, and the other encoding a protein conferring the additional functionalities to exosomes. In some aspects, the producer cell is further modified to comprise one, two, three, four, five, six, seven, eight, nine, or ten or more additional exogenous sequences.

In some aspects, EVs, e.g., exosomes, of the present disclosure (e.g., surface-engineered and/or lumen-engineered exosomes) can be produced from a cell transformed with a sequence encoding a full-length, mature scaffold moiety disclosed herein or a scaffold moiety linked to An IL-12 moiety. Any of the scaffold moieties described herein can be expressed from a plasmid, an exogenous sequence inserted into the genome or other exogenous nucleic acid, such as a synthetic messenger RNA (mRNA).

### V. Pharmaceutical Compositions

Provided herein are pharmaceutical compositions comprising an EV, e.g., exosome, of the present disclosure having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of extracellular vesicles. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

In some aspects, a pharmaceutical composition comprises one or more therapeutic agents and an exosome described herein. In certain aspects, the EVs, e.g., exosomes, are co-administered with one or more additional therapeutic agents, e.g., an anti-neoplastic agent, in a pharmaceutically acceptable carrier. In some aspects, the pharmaceutical composition comprising the EV, e.g., exosome is administered prior to administration of the additional therapeutic agent, e.g., the anti-neoplastic agent. In other aspects, the pharmaceutical composition comprising the EV, e.g., exosome, is administered after the administration of the additional therapeutic agent, e.g., the anti-neoplastic agent. In further aspects, the pharmaceutical composition comprising the EV, e.g., exosome, is administered concurrently with the additional therapeutic agent, e.g., the anti-neoplastic agent.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the extracellular vesicles described herein, use thereof in the compositions is contemplated. Supplementary therapeutic agents can also be incorporated into the compositions. Typically, a pharmaceutical composition is formulated to be compatible with its intended route of administration. The EVs, e.g., exosomes, can be administered by parenteral, topical, intravenous, oral, subcutaneous, intra-arterial, intradermal, transdermal, rectal, intracranial, intraperitoneal, intranasal, intratumoral, intramuscular route or as inhalants. In certain aspects, the pharmaceutical composition comprising exosomes is administered intravenously, e.g. by injection. The EVs, e.g., exosomes, can optionally be administered in combination with other therapeutic agents that are at least partly effective in treating the disease, disorder or condition for which the EVs, e.g., exosomes, are intended.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as mannitol, sorbitol, and sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the EVs, e.g., exosomes, in an effective amount and in an appropriate solvent with one or more ingredients enumerated herein or known in the art, as desired. Generally, dispersions are prepared by incorporating the EVs, e.g., exosomes, into a sterile vehicle that contains a basic dispersion medium and any desired other ingredients. In the case of sterile powders for the preparation of sterile injectable solutions, methods of preparation are vacuum drying and freeze-drying that yield a powder of the active ingredient plus any additional desired ingredient from a previously sterile-filtered solution thereof. The EVs, e.g., exosomes, can be administered in the form of a depot injection or implant preparation which can be formulated in such a manner to permit a sustained or pulsatile release of the EV, e.g., exosome.

Systemic administration of compositions comprising exosomes can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects the pharmaceutical composition comprising EVs, e.g., exosomes is administered intravenously into a subject that would benefit from the pharmaceutical composition. In certain other aspects, the composition is administered to the lymphatic system, e.g., by intralymphatic injection or by intranodal injection (see e.g., Senti et al., PNAS 105(46): 17908 (2008)), or by intramuscular injection, by subcutaneous administration, by intratumoral injection, by direct injection into the thymus, or into the liver.

In certain aspects, the pharmaceutical composition comprising exosomes is administered as a liquid suspension. In certain aspects, the pharmaceutical composition is administered as a formulation that is capable of forming a depot following administration. In certain preferred aspects, the depot slowly releases the EVs, e.g., exosomes, into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the EVs, e.g., exosomes, described herein and optionally a pharmaceutically active or therapeutic agent. The therapeutic agent can be a biological agent, a small molecule agent, or a nucleic acid agent.

Dosage forms are provided that comprise a pharmaceutical composition comprising the EVs, e.g., exosomes, described herein. In some aspects, the dosage form is formulated as a liquid suspension for intravenous injection. In some aspects, the dosage form is formulated as a liquid suspension for intratumoral injection.

In certain aspects, the preparation of exosomes is subjected to radiation, e.g., X rays, gamma rays, beta particles, alpha particles, neutrons, protons, elemental nuclei, UV rays in order to damage residual replication-competent nucleic acids.

In certain aspects, the preparation of exosomes is subjected to gamma irradiation using an irradiation dose of more than 1, 5, 10, 15, 20, 25, 30, 35, 40, 50, 60, 70, 80, 90, 100, or more than 100 kGy.

In certain aspects, the preparation of exosomes is subjected to X-ray irradiation using an irradiation dose of more than 0.1, 0.5, 1, 5, 10, 15, 20, 25, 30, 35, 40, 50, 60, 70, 80, 90, 100, 200, 300, 400, 500, 600, 700, 800, 900, 1000, 2000, 3000, 4000, 5000, 6000, 7000, 8000, 9000, 10000, or greater than 10000 mSv.

In some aspects, the exosome is in a pharmaceutical composition disclosed in International Publication No. WO 2021/062317, which is incorporated by reference herein in its entirety. In some aspects, the composition comprises:
(a) Extracellular vesicles comprising an IL-12 moiety disclosed herein;
(b) Sucrose at a concentration of about 146 mM;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an IL-12 moiety disclosed herein;
(b) Sucrose at a concentration of about 5%;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

### VI. Kits

Also provided herein are kits comprising one or more exosomes described herein. In some aspects, provided herein is a pharmaceutical pack or kit comprising one or more containers filled with one or more of the ingredients of the pharmaceutical compositions described herein, such as one or more exosomes provided herein, optional an instruction for use. In some aspects, the kits contain a pharmaceutical composition described herein and any prophylactic or therapeutic agent, such as those described herein. In some aspects, the kit further comprises instructions to administer the EV according to any method disclosed herein.

### VI. Methods of Producing EVs

In some aspects, the present disclosure is also directed to methods of producing EVs described herein. In some aspects, the method comprises: obtaining the EV, e.g., exosome from a producer cell, wherein the producer cell contains one or more components of the EV, e.g., exosome (e.g., An IL-12 moiety; and optionally isolating the obtained EV, e.g., exosome. In some aspects, the method comprises: modifying a producer cell by introducing one or more components of an EV disclosed herein (e.g., An IL-12 moiety); obtaining the EV, e.g., exosome from the modified producer cell; and optionally isolating the obtained EV, e.g., exosome. In further aspects, the method comprises: obtaining an EV from a producer cell; isolating the obtained EV; and modifying the isolated EV. In certain aspects, the method further comprises formulating the isolated EV into a pharmaceutical composition.

### VI.A. Methods of Modifying a Producer Cell

As described supra, in some aspects, a method of producing an EV comprises modifying a producer cell with one or more moieties (e.g., an IL-12 moiety). In certain aspects, the one or more moieties comprise an IL-12 moiety. In some aspects, the one or more moieties further comprise a scaffold moiety disclosed herein (e.g., Scaffold X or Scaffold Y).

In some aspects, the producer cell can be a mammalian cell line, a plant cell line, an insect cell line, a fungi cell line, or a prokaryotic cell line. In certain aspects, the producer cell is a mammalian cell line. Non-limiting examples of mammalian cell lines include: a human embryonic kidney (HEK) cell line, a Chinese hamster ovary (CHO) cell line, an HT-1080 cell line, a HeLa cell line, a PERC-6 cell line, a CEVEC cell line, a fibroblast cell line, an amniocyte cell line, an epithelial cell line, a mesenchymal stem cell (MSC) cell line, and combinations thereof. In certain aspects, the mammalian cell line comprises HEK-293 cells, BJ human foreskin fibroblast cells, fHDF fibroblast cells, AGE.HN® neuronal precursor cells, CAP® amniocyte cells, adipose mesenchymal stem cells, RPTEC/TERT1 cells, or combinations thereof. In some aspects, the producer cell is a primary cell. In certain aspects, the primary cell can be a primary mammalian cell, a primary plant cell, a primary insect cell, a primary fungi cell, or a primary prokaryotic cell.

In some aspects, the producer cell is not an immune cell, such as an antigen presenting cell, a T cell, a B cell, a natural killer cell (NK cell), a macrophage, a T helper cell, or a regulatory T cell (Treg cell). In other aspects, the producer cell is not an antigen presenting cell (e.g., dendritic cells, macrophages, B cells, mast cells, neutrophils, Kupffer-Browicz cell, or a cell derived from any such cells).

In some aspects, the one or more moieties can be a transgene or mRNA, and introduced into the producer cell by transfection, viral transduction, electroporation, extrusion, sonication, cell fusion, or other methods that are known to the skilled in the art.

In some aspects, the one or more moieties is introduced to the producer cell by transfection. In some aspects, the one or more moieties can be introduced into suitable producer cells using synthetic macromolecules, such as cationic lipids and polymers (Papapetrou et al., Gene Therapy 12: S118-S130 (2005)). In some aspects, the cationic lipids form complexes with the one or more moieties through charge interactions. In some of these aspects, the positively charged complexes bind to the negatively charged cell surface and are taken up by the cell by endocytosis. In some other aspects, a cationic polymer can be used to transfect producer cells. In some of these aspects, the cationic polymer is polyethylenimine (PEI). In certain aspects, chemicals such as calcium phosphate, cyclodextrin, or polybrene, can be used to introduce the one or more moieties to the producer cells. The one or more moieties can also be introduced into a producer cell using a physical method such as particle-mediated transfection, “gene gun”, biolistics, or particle bombardment technology (Papapetrou et al., Gene Therapy 12: S118-S130 (2005)). A reporter gene such as, for example, beta-galactosidase, chloramphenicol acetyltransferase, luciferase, or green fluorescent protein can be used to assess the transfection efficiency of the producer cell.

In certain aspects, the one or more moieties are introduced to the producer cell by viral transduction. A number of viruses can be used as gene transfer vehicles, including Maloney murine leukemia virus (MMLV), adenovirus, adeno-associated virus (AAV), herpes simplex virus (HSV), lentiviruses, and spumaviruses. The viral mediated gene transfer vehicles comprise vectors based on DNA viruses, such as adenovirus, adeno-associated virus and herpes virus, as well as retroviral based vectors.

In certain aspects, the one or more moieties are introduced to the producer cell by electroporation. Electroporation creates transient pores in the cell membrane, allowing for the introduction of various molecules into the cell. In some aspects, DNA and RNA as well as polypeptides and non-polypeptide therapeutic agents can be introduced into the producer cell by electroporation.

In certain aspects, the one or more moieties introduced to the producer cell by microinjection. In some aspects, a glass micropipette can be used to inject the one or more moieties into the producer cell at the microscopic level.

In certain aspects, the one or more moieties are introduced to the producer cell by extrusion.

In certain aspects, the one or more moieties are introduced to the producer cell by sonication. In some aspects, the producer cell is exposed to high intensity sound waves, causing transient disruption of the cell membrane allowing loading of the one or more moieties.

In certain aspects, the one or more moieties are introduced to the producer cell by cell fusion. In some aspects, the one or more moieties are introduced by electrical cell fusion. In other aspects, polyethylene glycol (PEG) is used to fuse the producer cells. In further aspects, sendai virus is used to fuse the producer cells.

In some aspects, the one or more moieties are introduced to the producer cell by hypotonic lysis. In such aspects, the producer cell can be exposed to low ionic strength buffer causing them to burst allowing loading of the one or more moieties. In other aspects, controlled dialysis against a hypotonic solution can be used to swell the producer cell and to create pores in the producer cell membrane. The producer cell is subsequently exposed to conditions that allow resealing of the membrane.

In some aspects, the one or more moieties are introduced to the producer cell by detergent treatment. In certain aspects, producer cell is treated with a mild detergent which transiently compromises the producer cell membrane by creating pores allowing loading of the one or more moieties. After producer cells are loaded, the detergent is washed away thereby resealing the membrane.

In some aspects, the one or more moieties introduced to the producer cell by receptor mediated endocytosis. In certain aspects, producer cells have a surface receptor which upon binding of the one or more moieties induces internalization of the receptor and the associated moieties.

In some aspects, the one or more moieties are introduced to the producer cell by filtration. In certain aspects, the producer cells and the one or more moieties can be forced through a filter of pore size smaller than the producer cell causing transient disruption of the producer cell membrane and allowing the one or more moieties to enter the producer cell.

In some aspects, the producer cell is subjected to several freeze thaw cycles, resulting in cell membrane disruption allowing loading of the one or more moieties.

### VII.B. Methods of Modifying EV, e.g., Exosome

In some aspects, a method of producing an EV, e.g., exosome, comprises modifying the isolated EV by directly introducing one or more moieties into the EVs. In certain aspects, the one or more moieties comprise an IL-12 moiety. In some aspects, the one or more moieties comprise a scaffold moiety disclosed herein (e.g., Scaffold X or Scaffold Y).

In certain aspects, the one or more moieties are introduced to the EV by transfection. In some aspects, the one or more moieties can be introduced into the EV using synthetic macromolecules such as cationic lipids and polymers (Papapetrou et al., Gene Therapy 12: S118-S130 (2005)). In certain aspects, chemicals such as calcium phosphate, cyclodextrin, or polybrene, can be used to introduce the one or more moieties to the EV.

In certain aspects, the one or more moieties are introduced to the EV by electroporation. In some aspects, EVs are exposed to an electrical field which causes transient holes in the EV membrane, allowing loading of the one or more moieties.

In certain aspects, the one or more moieties are introduced to the EV by microinjection. In some aspects, a glass micropipette can be used to inject the one or more moieties directly into the EV at the microscopic level.

In certain aspects, the one or more moieties are introduced to the EV by extrusion.

In certain aspects, the one or more moieties are introduced to the EV by sonication. In some aspects, EVs are exposed to high intensity sound waves, causing transient disruption of the EV membrane allowing loading of the one or more moieties.

In some aspects, one or more moieties can be conjugated to the surface of the EV. Conjugation can be achieved chemically or enzymatically, by methods known in the art.

In some aspects, the EV comprises one or more moieties that are chemically conjugated. Chemical conjugation can be accomplished by covalent bonding of the one or more moieties to another molecule, with or without use of a linker. The formation of such conjugates is within the skill of artisans and various techniques are known for accomplishing the conjugation, with the choice of the particular technique being guided by the materials to be conjugated. In certain aspects, polypeptides are conjugated to the EV. In some aspects, non-polypeptides, such as lipids, carbohydrates, nucleic acids, and small molecules, are conjugated to the EV.

In some aspects, the one or more moieties are introduced to the EV by hypotonic lysis. In such aspects, the EVs can be exposed to low ionic strength buffer causing them to burst allowing loading of the one or more moieties. In other aspects, controlled dialysis against a hypotonic solution can be used to swell the EV and to create pores in the EV membrane. The EV is subsequently exposed to conditions that allow resealing of the membrane.

In some aspects, the one or more moieties are introduced to the EV by detergent treatment. In certain aspects, extracellular vesicles are treated with a mild detergent which transiently compromises the EV membrane by creating pores allowing loading of the one or more moieties. After EVs are loaded, the detergent is washed away thereby resealing the membrane.

In some aspects, the one or more moieties are introduced to the EV by receptor mediated endocytosis. In certain aspects, EVs have a surface receptor which upon binding of the one or more moieties induces internalization of the receptor and the associated moieties.

In some aspects, the one or more moieties are introduced to the EV by mechanical firing. In certain aspects, extracellular vesicles can be bombarded with one or more moieties attached to a heavy or charged particle such as gold microcarriers. In some of these aspects, the particle can be mechanically or electrically accelerated such that it traverses the EV membrane.

In some aspects, extracellular vesicles are subjected to several freeze thaw cycles, resulting in EV membrane disruption allowing loading of the one or more moieties.

### VII.C. Methods of Isolating EV, e.g., Exosome

In some aspects, methods of producing EVs disclosed herein comprises isolating the EV from the producer cells. In certain aspects, the EVs released by the producer cell into the cell culture medium. It is contemplated that all known manners of isolation of EVs are deemed suitable for use herein. For example, physical properties of EVs can be employed to separate them from a medium or other source material, including separation on the basis of electrical charge (e.g., electrophoretic separation), size (e.g., filtration, molecular sieving, etc.), density (e.g., regular or gradient centrifugation), Svedberg constant (e.g., sedimentation with or without external force, etc.). Alternatively, or additionally, isolation can be based on one or more biological properties, and include methods that can employ surface markers (e.g., for precipitation, reversible binding to solid phase, FACS separation, specific ligand binding, non-specific ligand binding, affinity purification etc.).

Isolation and enrichment can be done in a general and non-selective manner, typically including serial centrifugation. Alternatively, isolation and enrichment can be done in a more specific and selective manner, such as using EV or producer cell-specific surface markers. For example, specific surface markers can be used in immunoprecipitation, FACS sorting, affinity purification, and magnetic separation with bead-bound ligands.

In some aspects, size exclusion chromatography can be utilized to isolate the EVs. Size exclusion chromatography techniques are known in the art. Exemplary, non-limiting techniques are provided herein. In some aspects, a void volume fraction is isolated and comprises the EVs of interest. Further, in some aspects, the EVs can be further isolated after chromatographic separation by centrifugation techniques (of one or more chromatography fractions), as is generally known in the art. In some aspects, for example, density gradient centrifugation can be utilized to further isolate the extracellular vesicles. In certain aspects, it can be desirable to further separate the producer cell-derived EVs from EVs of other origin. For example, the producer cell-derived EVs can be separated from non-producer cell-derived EVs by immunosorbent capture using an antigen antibody specific for the producer cell.

In some aspects, the isolation of EVs can involve combinations of methods that include, but are not limited to, differential centrifugation, size-based membrane filtration, immunoprecipitation, FACS sorting, and magnetic separation.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (TRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); Crooke, Antisense drug Technology: Principles, Strategies and Applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1

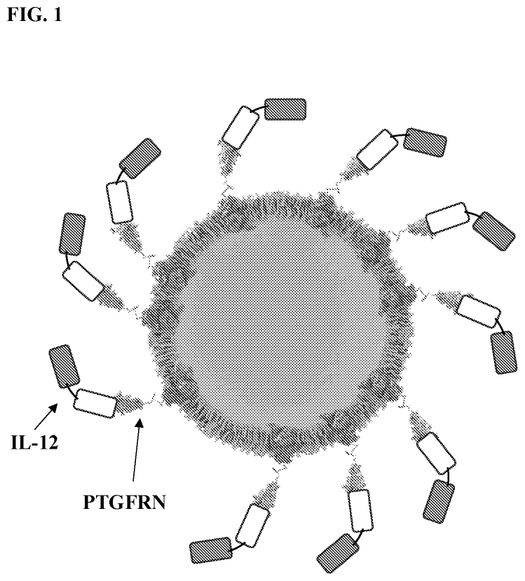

Exosomes with surface-displayed IL-12 were prepared by expressing in exosome-producing cells a fusion construct comprising a single peptide IL-12 (p29 linked by a peptide linker p40) linked to PTGFRN (FIG. 1). Potency was assessed in vitro using human PBMCs or murine splenocytes and in vivo using mouse subcutaneous tumor models. Local versus systemic pharmacology was determined with intratumoral injection in mice and subcutaneous injection in monkeys. All studies were benchmarked against recombinant IL-12 (rIL-12).

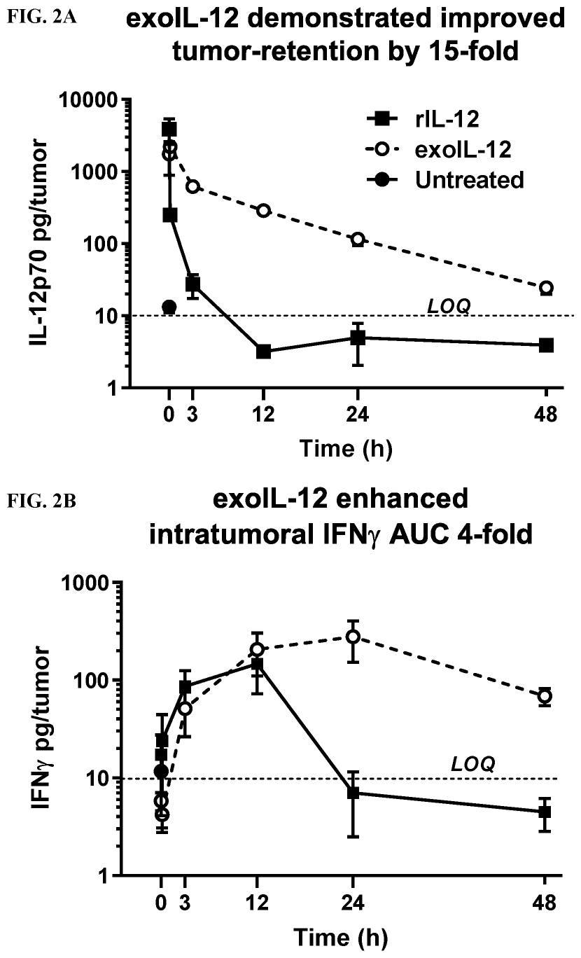

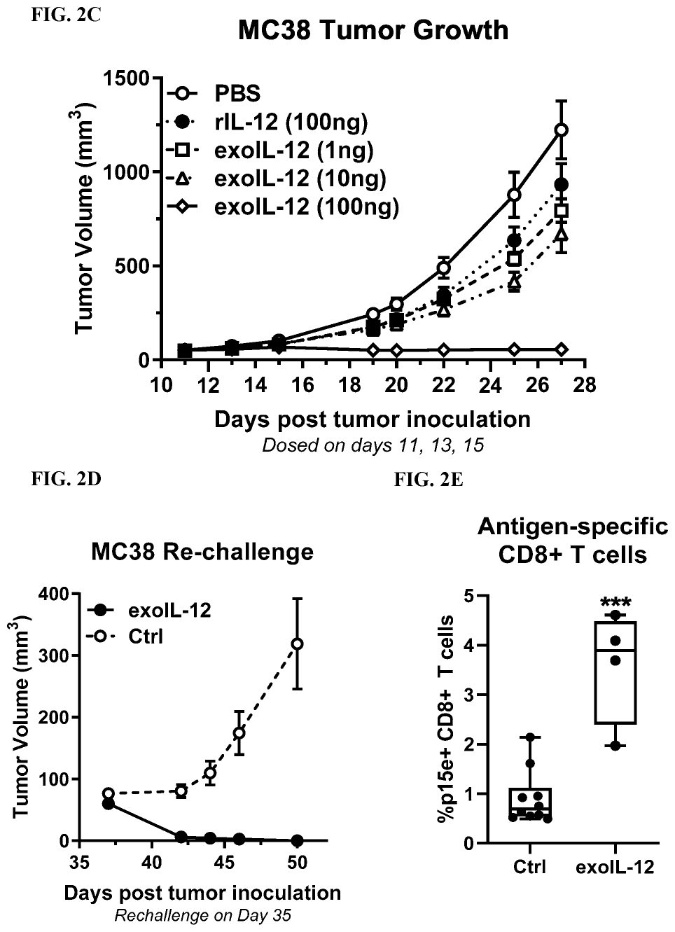

In an MC38 mouse tumor model, intratumoral administration of exosomes with surface-displayed IL-12 (exoIL-12) showed enhanced PK and sustained PD as compared to free recombinant IL-12 and untreated controls. Mice administered exoIL-12 showed increased tumor retention (of about 15-fold) as measured by IL-12p70 concentration per tumor at 3, 12, 24, and 48 hours post administration, compared with free recombinant IL-12 (FIG. 2A). In addition, exoIL-12 administration led to enhanced intratumoral IFN-gamma AUC by about 4-fold as compared to recombinant IL-12 (FIG. 2B). Further, intratumoral administration of exoIL-12 led to a dose dependent reduction in MC38 tumor growth in mice. ExoIL-12 was 100-fold more potent than rIL-12 in tumor growth inhibition, with mice receiving 100 ng exoIL-12 showing little to no tumor growth (FIG. 2C). In the MC38 tumor model, complete responses were observed in 63% of mice treated with exoIL-12; in contrast, rIL-12 resulted in 0% complete responses at an equivalent IL-12 dose. This correlated with dose-dependent increases in tumor antigen-specific CD8+ T cells, which increased nearly 4-fold in exoIL-12 treated mice (FIG. 2E).

Suppression of tumor growth was again observed following MC38 re-challenge (FIG. 2D). Re-challenge studies of exoIL-12 complete responder mice showed no tumor regrowth and depletion of CD8+ T cells completely abrogated antitumor activity of exoIL-12. Following intratumoral administration, exoIL-12 exhibited 10-fold higher intratumoral exposure than rIL-12 and prolonged IFNγ production up to 48 hr. Retained local pharmacology of exoIL-12 was further confirmed using subcutaneous injections in non-human primates.

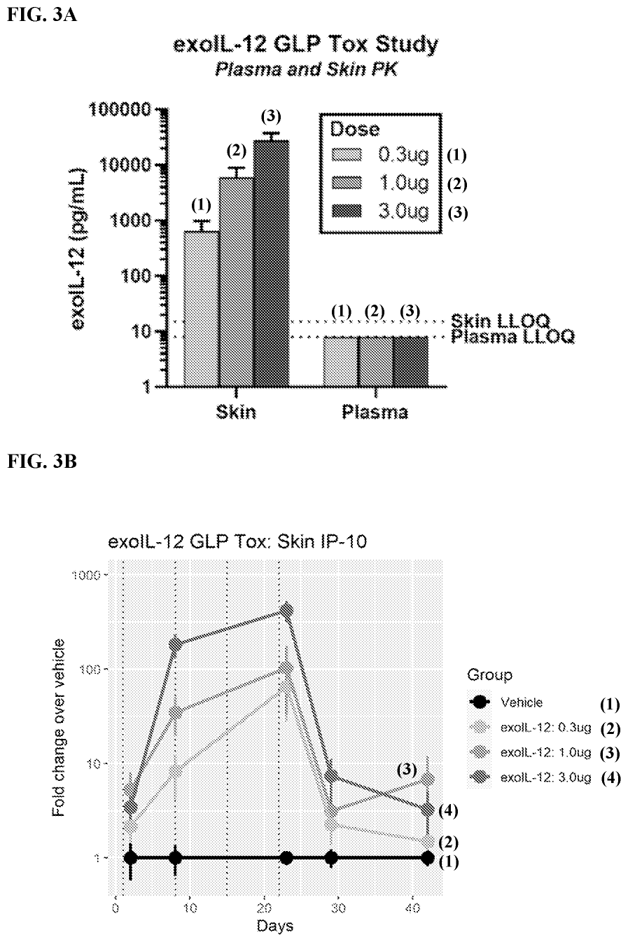

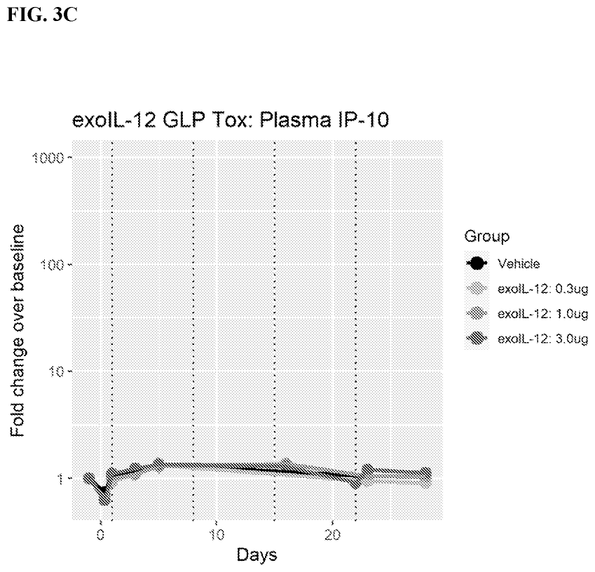

Toxicology analysis revealed that the highest dose tested, 3 μg exoIL-12, was NOAEL (no observable adverse effect level), showing limited plasma levels and dose dependent tissue levels (FIG. 3A). CXCL10/IP-10 expression was observed to be sustained in the skin after a single dose but was undetectable in plasma (FIGS. 3B-3C).

Tumor-restricted pharmacology of exoIL-12 results in superior in vivo efficacy and immune memory without systemic IL-12 exposure and related toxicity. As such, exoIL-12 overcomes key limitations of rIL-12.

### Example 2

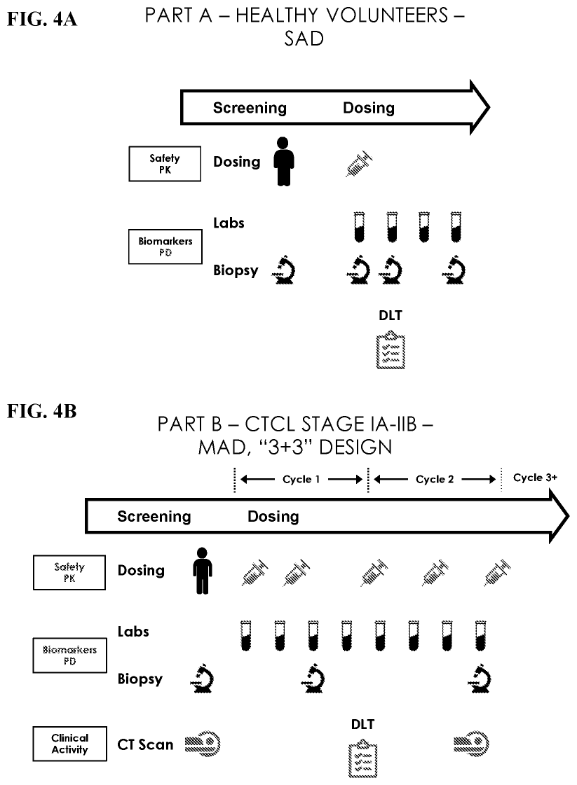

A clinical study will be conducted to test the safety and efficacy of treating a cancer in a human subject by administering engineered exosomes comprising surface displayed IL-12 (FIGS. 4A-4B). In part A, healthy volunteers will be administered varying doses of exoIL-12 and monitored of adverse events and biomarkers (FIG. 4A). In part B, subjects diagnosed with CTCL (stage IA-IIB) will be administered various doses of exoIL-12 and monitored for safety and biomarkers (FIG. 41B). Clinical activity will be monitored by one or more CT scan. Subjects having CTCL, TNBC, melanoma, GBM, MCC, and/or Kaposi sarcoma will be eligible for part B ofthe trial.

### Example 3

A Phase I trial of exoxL-12 was conducted in healthy volunteers (Table 4). A total of five cohorts each with five subjects, randomized 3:2 active drug to placebo, were enrolled and dosed in the first part of the study. Each cohort received a subcutaneously administered single ascending dose of exoIL-12: 0.3 μg, 1.0 μg, 3.0 μg, 6.0 μg, and 12.0 μg.

TABLE 4
Patient Demographics.

| | | | Race | | | | | | | |
|---|---|---|---|---|---|---|---|---|---|---|
| | Age | | A: 4/25 (16%) | | Weight (kg) | | Height (cm) | | BMI | |
| | 27 | | B: 6/25 (24%) | | 69.7 | | 177.2 | | 22.1 | |
| All | (18; 40) | | C: 15/25 (60%) | | (55; 83) | | (167; 190) | | (18.3; 24.9) | |
| N = 25 | exoIL-12 | Placebo | exoIL-12 | Placebo | exoIL-12 | Placebo | exoIL-12 | Placebo | exoIL-12 | Placebo |
| Cohort 1 - | 32 | 26 | C (2), | C (2) | 70 | 70.5 | 177.3 | 177 | 22.3 | 22.5 |
| 0.3 μg | (22; 37) | (25; 27) | B (1) | | (67; 73.8) | (65.4; 75.7) | (176; 178) | (171; 183) | (21.6; 23.3) | (22.4; 22.6) |
| N = 5 | | | | | | | | | | |
| Cohort 2 - | 23 | 19 | B (3) | C (2) | 67.8 | 80.6 | 177 | 190 | 21.6 | 22.3 |
| 1.0 μg | (19; 29) | (19; 19) | | | (57.2; 77.2) | (78.1; 83.2) | (168:185) | (190; 190) | (20.3:22.6) | (21.6; 23) |
| N = 5 | | | | | | | | | | |
| Cohort 3 - | 20 | 26 | C (2), | A (2) | 69.4 | 65.3 | 176.3 | 175 | 22.2 | 21.3 |
| 3.0 μg | (18; 22) | (20; 31) | A (1) | | (60.7; 82.8) | (54.9; 75.7) | (169; 183) | (173; 177) | (20.7; 24.7) | (18.3; 24.2) |
| N = 5 | | | | | | | | | | |
| Cohort 4 - | 31 | 27.5 | A (1), | C (2) | 70.3 | 71.6 | 179.7 | 178 | 21.6 | 22.6 |
| 6.0 μg | (24; 36) | (21; 34) | B (1), | | (56.1; 79.7) | (68.7; 74.5) | (170; 190) | (176; 180) | (19.4; 23.4) | (22.2; 23) |
| N = 5 | | | C (1) | | | | | | | |
| Cohort 5 - | 32.7 | 28 | B (1), | C (2) | 64.3 | 69.9 | 173 | 170.5 | 21.6 | 24.1 |
| 12.0 μg | (25; 40) | (21; 35) | C (2) | | (61.9; 68.5) | (67.7; 72) | (167; 176) | (170; 171) | (20; 24.6) | (23.2; 24.9) |
| N = 5 | | | | | | | | | | |

Numbers listed as Average (Min; Max);
A: Asian,
B: Black or African American,
C: Caucasian

The data show that the primary objectives were met in the initial part of the Phase 1 trial. In this randomized, placebo controlled, double-blind study, exoIL-12 demonstrated a favorable safety and tolerability profile, with no local or systemic treatment-related adverse events and no detectable systemic exposure of IL-12. No treatment-related adverse events were observed throughout 10 days of follow-up. In particular, no chills, fever, fatigue, dizziness, myalgia, headache or back pain were reported. These symptoms have been observed in previous clinical studies of subcutaneously administered rIL-12 at comparable doses to those used in this study of exoIL-12 (ranging from 2 μg to 12 μg). Gokhale M S, et al. Exp Hematol Oncol. 2014; 3(1):11. Published 2014 Apr. 11. doi:10.1186/2162-3619-3-11

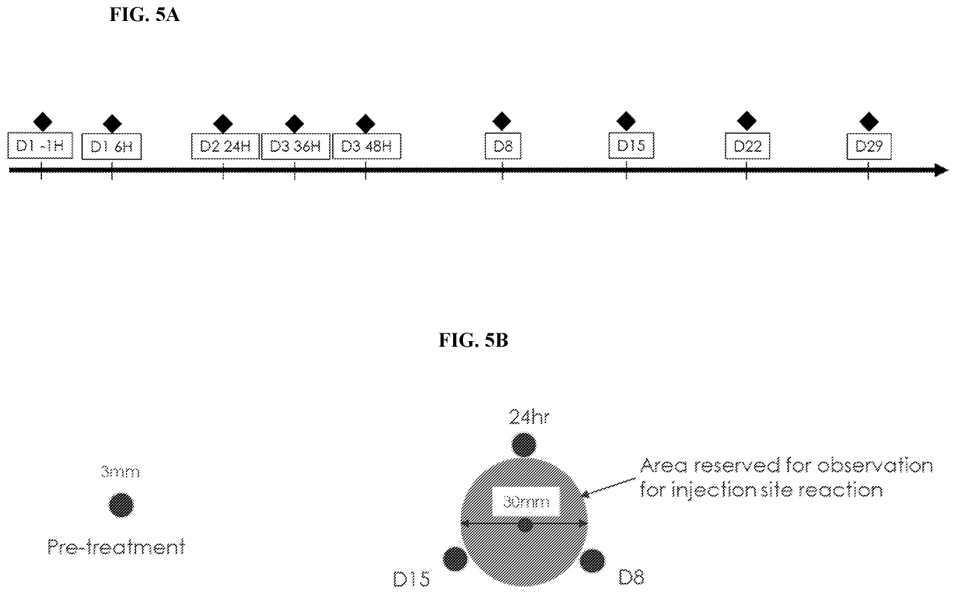

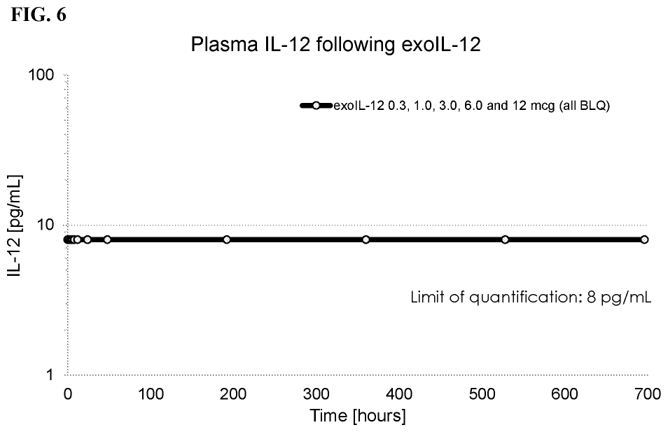

Plasma and skin biopsy cores were collected at various time points (FIGS. 5A-5B). Plasma pharmacokinetic (PK) measurements showed no systemic exposure with levels of IL-12 below the limit of quantification at all doses tested (FIG. 6). In contrast, previous clinical studies with rIL-12 showed dose-dependent systemic exposure with dosages of 5 μg and 12 μg resulting in Cmax plasma levels of approximately 15 μg/ml to 45 μg/ml within 6 to 12 hours after dosing. See Gokhale M S, et al., 2014 (supra). These data demonstrate the potential advantage of exoIL-12, e.g., lower systemic toxicity, compared to recombinant IL-12 (rIL-12)-based therapies that have generally demonstrated limitations due to significant safety and tolerability concerns. To overcome these limitations, exoIL-12 was designed to facilitate dose control of IL-12 and limit systemic exposure and associated toxicity by localizing IL-12 in the tumor microenvironment (TME) in order to potentially expand the therapeutic index.

Confirmation of local retention of IL-12 is assessed by measurement of IL-12 levels in skin punch biopsies pre-treatment and at 24 hrs, day 8 and day 15 post treatment. Without being bound by any theory, it is expected that no difference will be found in systemic IFNγ and IP-10 between exoIL-12 and placebo, consistent with an absence of systemic IL-12 exposure.

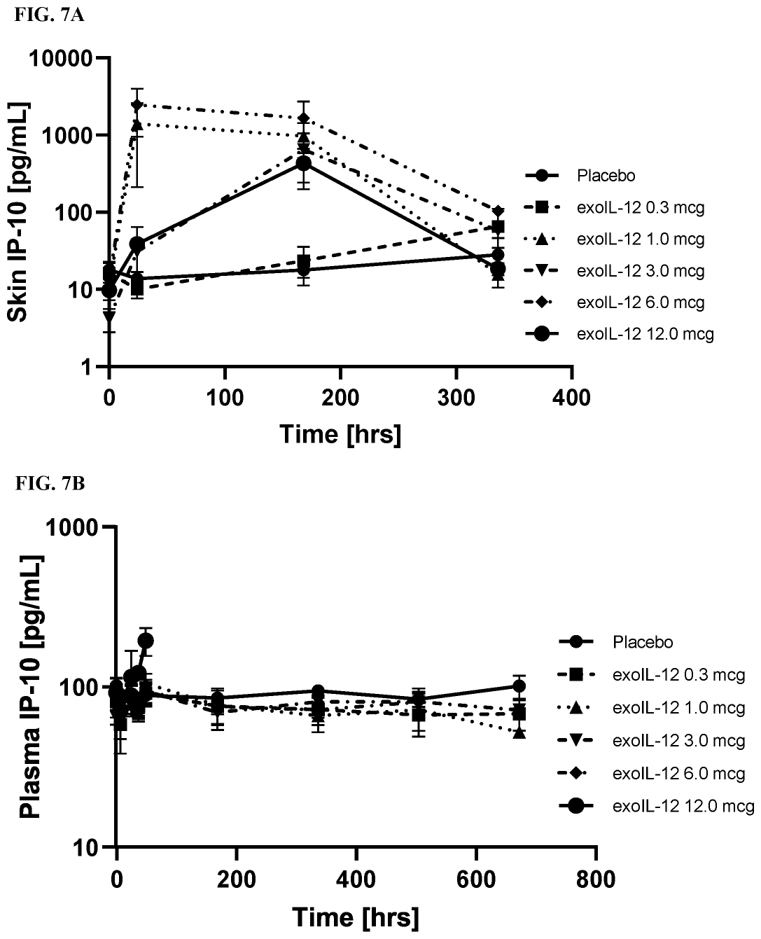

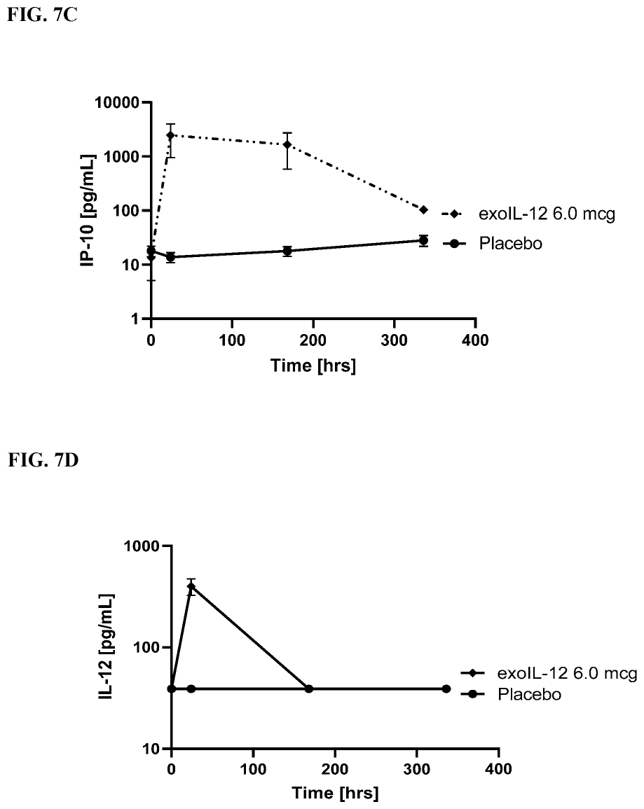

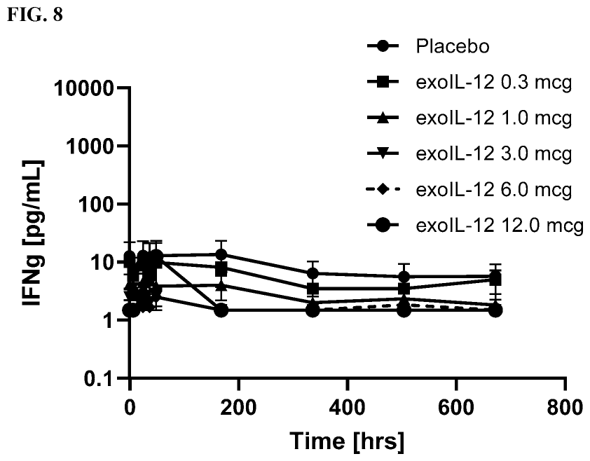

Local pharmacodynamic effects are assessed by quantifying IP-10 levels in the serial skin punch biopsies and with exploratory gene expression analyses. Skin biopsies were obtained using Stiefel Biopsy Punch (3 mm and scalpel), penetrating the epidermis and dermis and reaching upper sub-cutaneous tissue. Biopsies were taken at least 30 mm away from the injection site of exoIL-12 (FIG. 5B). Potent, durable IP-10 effects were observed in skin biopsies following exoIL-12 administration at all dosing levels (0.3 μg, 1.0 μg, 3.0 μg, 6.0 μg, or 12.0 μg exoIL-12; FIG. 7A). The highest average IP-10 levels in skin biopsies were observed in subjects administered the 6.0 μg dose (FIG. 7A, diamonds). Conversely, there was no increase in plasma IP-10 observed in subjects administered 0.3 μg, 1.0 μg, 3.0 μg, or 6.0 μg exoIL-12, although there was a slight increase in plasma IP-10 at exoIL-12 12.0 μg on Day 3 (FIG. 7B). High levels of IL-12 and IP-10 were observed in skin biopsies of subjects administered 6.0 μg exoIL-12, and activity was measurable on Day 8 (FIGS. 7C-7D). Further, there was no increase in plasma IFNγ observed in subjects administered 0.3 μg, 1.0 μg, 3.0 μg, 6.0 μg, or 12.0 μg exoIL-12 (FIG. 8), whereas an increase in plasma IFNγ has been observed previously following administration of rIL-12 (12.0 μg). These results show the desired localization and retention of IL-12 at the injection site for at least 24 hours, as well as prolonged IP-10 production for 8-15 days depending upon dose; and these data are supportive of every other week dosing in part B of the study in subjects administered 6.0 μg exoIL-12.

These results indicate that exoIL-12 was well tolerated at SADs from 0.3 to 12.0 g in healthy volunteers. No systemic exposure of IL-12 was observed in subjects administered exoIL-12, in contrast to historical results for subjects receiving comparable dosages of rIL-12. exoIL-12 was detectable in skin at 6 μg, suggesting retention of exoIL-12 at the injection site. ExoIL-12 at 1.0 μg to 12.0 μg showed potent skin IP-10 levels, with the highest levels observed following administration of 6.0 μg exoIL-12. These data support continuing the study in part B using a starting dose of 6.0 μg every other week. The next portion of the Phase 1 clinical trial will proceed, evaluating the safety and efficacy of exoIL-12 in cutaneous T Cell lymphoma (CTCL) patients at the dose of 6 μg.

### Example 4

A clinical trial will be conducted to study the safety and efficacy of administering exoIL-12 to cancer subjects, including subjects afflicted with cutaneous T-cell lymphoma (CTCL), melanoma, triple negative breast cancer (TNBC), glioblastoma, Merkel cell carcinoma (MCC), Kaposi sarcoma, basal cell carcinoma (BCC), bladder cancer, or a combination thereof. Subjects will be administered 6 μg exoIL-12 or 12 μg exoIL-12 once every two weeks. Subjects will be monitored for response rate, overall survival (OS), progression free survival (PFS), and the occurrence of adverse events.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the invention(s). Many variations will become apparent to those skilled in the art upon review of this specification.

1. A method of treating cutaneous T-cell lymphoma (CTCL), triple negative breast cancer (TNBC), glioblastoma, Merkel cell carcinoma (MCC), and/or Kaposi sarcoma in a subject in need thereof comprising administering an extracellular vesicle (EV) comprising Interleukin-12 (IL-12), wherein the IL-12 or a fragment thereof comprises an amino acid sequence having at least 90%, at least 95%, at least 96%, at least 97%, at least 98%, at least 99%, or about 100% sequence identity to SEQ ID NO: 2, 3 or 4.

2. (canceled)

3. The method of claim 1, wherein the CTCL is at stage IA-IIB.

4-7. (canceled)

8. The method of claim 1, wherein the administration of the EV results in at least about 20%, at least about 25%, at least about 30%, at least about 35%, at least about 40%, at least about 45%, at least about 50%, at least about 55%, at least about 56%, at least about 60%, at least about 70%, at least about 80%, at least about 90%, or about 100% objective response rate (ORR).

9. (canceled)

10. The method of claim 1, wherein the administration of the EV results in at least about 10%, at least about 20%, at least about 22%, at least about 25%, at least about 30%, at least about 35%, at least about 40%, at least about 45%, at least about 50%, at least about 55%, at least about 60%, at least about 70%, at least about 80%, at least about 90%, or about 100% complete response (CR).

11. (canceled)

12. The method of claim 1, wherein the EV further comprises a scaffold moiety.

13. The method of claim 12, wherein the IL-12 is linked to the scaffold moiety.

14. The method of claim 13, wherein the scaffold moiety is a Scaffold X.

15. The method of claim 14, wherein Scaffold X is a scaffold protein that is capable of anchoring the IL-12 on the exterior surface of the EV.

16. The method of claim 14, wherein Scaffold X is selected from the group consisting of prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), and any combination thereof.

17. (canceled)

18. The method of claim 16, wherein the scaffold moiety is the PTGFRN protein or a fragment thereof, having at least 90%, at least 95%, at least 96%, at least 97%, at least 98%, at least 99%, or about 100% sequence identity to SEQ ID NO: 1 or 33.

19. (canceled)

20. The method of claim 1, wherein the IL-12 is linked to the scaffold moiety by a linker, wherein the linker is a polypeptide or a non-polypeptide moiety.

21-25. (canceled)

26. The method of claim 20, wherein the IL-12 comprises p35 and p40 linked by a linker, wherein the linker is a GS linker, wherein the GS linker comprises (G4S)n or (G3S)n, wherein n is any integer between 1 and 10.

27-29. (canceled)

30. The method of claim 1, wherein the EV is administered parenterally, orally, intravenously, intramuscularly, intra-tumorally, intranasally, subcutaneously, or intraperitoneally.

31. The method of claim 1, wherein the EV is an exosome.

32. (canceled)

33. The method of claim 1, comprising further administering an additional therapeutic agent.

34. The method of claim 33, wherein the additional therapeutic agent is an anti-neoplastic agent.

35. The method of claim 34, wherein the anti-neoplastic agent is an immune checkpoint inhibitor.

36. The method of claim 35, wherein the immune checkpoint inhibitor comprises an anti-PD-1 antibody, an anti-PD-L1 antibody, an anti-LAG3 antibody, an anti-TIM3 antibody, an anti-CTLA4 antibody, an anti-TIGIT antibody, or any combination thereof.

37-40. (canceled)

41. The method of claim 1, wherein:
(i) the EV is administered at a therapeutically effective amount of at least about 0.3 μg, at least about 1 μg, at least about 2 μg, at least about 3 μg, at least about 4 μg, at least about 5 μg, at least about 6 μg, at least about 7 μg, at least about 8 μg, at least about 9 μg, at least about 10 μg, at least about 11 μg, or at least about 12 μg,
(ii) the therapeutically effective amount is administered in one or more doses,
(iii) the therapeutically effective amount exhibits less systemic toxicity in the subject compared to the administration of the same dose of recombinant IL-12, or
(iv) any combination of (i)-(iii).

42-45. (canceled)

46. The method of claim 1, wherein the EV is administered at a therapeutically effective amount of about 5 μg to about 7 μg, once about every week, once about every other week, once about every three weeks, once about every four weeks, once about every 10 to 18 days, once about every 12 to about 16 days, once about every 14 to about 21 days, once about every 10 to 14 days, or once about every 14 to about 18 days.

47. (canceled)
